SEQUENCE LISTING (8 sequences)

SEQ ID NO: 1 - DNA, 234 bp, Agrostis stolonifera
ctacatcaacaagcccaagatgcggcactacgtgcactgctacgcgctgcactgcctggacgaggaggcctccgacgcgctgcgcagggcgtacaaggcccgcggcgagaacgtcggcgcctggaggcaggcgtgctacgcgccgctggtggacatctccgccaggcacggcttcgacgtcgacgccgtcttcgccgcgcacccgcgcctcgccatctggtacgtgcccaccag

SEQ ID NO: 2 - DNA, 234 bp, Agrostis stolonifera
ctggtgggcacgtaccagatggcgaggcgcgggtgcgcggcgaagacggcgtcgacgtcgaagccgtgcctggcggagatgtccaccagcggcgcgtagcacgcctgcctccaggcgccgacgttctcgccgcgggccttgtacgccctgcgcagcgcgtcggaggcctcctcgtccaggcagtgcagcgcgtagcagtgcacgtagtgccgcatcttgggcttgttgatgtag

SEQ ID NO: 3 - DNA, 21 bp, Artificial
  PCR primer
atgaattttactggcttccat

SEQ ID NO: 4 - DNA, 21 bp, Artificial
  PCR primer
aacccacatgttcacggcgga

SEQ ID NO: 5 - DNA, 24 bp, Artificial
  PCR primer
ttcacattctcttcctcgtgttca

SEQ ID NO: 6 - DNA, 24 bp, Artificial
  PCR primer
tcgtagttagctccggtacaagtg

SEQ ID NO: 7 - protein, 47 aa, Litopenaeus setiferus
HSSGYTRPLRKPSRPIFIRPIGCDVCYGIPSSTARLCCFRYGDCCHL

SEQ ID NO: 8 - DNA, 144 bp, Artificial
  Modified L. setiferus Pen4-1 gene sequence for plant expression
cactcctccggctacaccaggcccctcaggaagccctccaggcccatcttcatcaggcccatcggctgcgacgtctgctacggcatcccctcctccaccgccaggctctgctgcttcaggtacggcgactgctgccacctctag

(19) United States
(12) Patent Application Publication
(10) Pub. No.: US 2011/0078820 A1
(43) Pub. Date: Mar. 31, 2011

(54) METHODS AND COMPOSITIONS FOR TRANSGENIC PLANTS PRODUCING ANTIMICROBIAL PEPTIDES FOR ENHANCED DISEASE RESISTANCE

(75) Inventors: Hong Luo, Clemson, SC (US); Man Zhou, Central, SC (US); Qian Hu, Clemson, SC (US)

(73) Assignee: Clemson University Research Foundation

(21) Appl. No.: 12/888,830

(22) Filed: Sep. 23, 2010

Related U.S. Application Data
(60) Provisional application No. 61/247,103, filed on Sep. 30, 2009.

(51) Int. Cl. C12N 15/82; C07H 21/00; C12N 5/10; A01H 5/00; A01H 5/10

(52) U.S. Cl. 800/279; 536/23.5; 435/419; 800/298; 800/301

(57) ABSTRACT

The present invention provides methods and compositions for producing transgenic plants having increased disease resistance resulting from the expression of exogenous nucleotide sequences encoding antimicrobial peptides.

### STATEMENT OF PRIORITY

The present application claims the benefit, under 35 U.S.C. §119(e), of U.S. Provisional Application Ser. No. 61/247,103, filed Sep. 30, 2009, the entire contents of which are incorporated herein by reference.

### STATEMENT OF GOVERNMENT SUPPORT

Aspects of this invention were supported by funding under Grant Nos. USDA-BRAGG 2005-39454-16551, 2007-33522-18489 and USDA CSREES SC-1700315. The United States Government has certain rights in this invention.

### FIELD OF THE INVENTION

The present invention relates to methods and compositions for producing transgenic plants with enhanced disease resistance.

### BACKGROUND OF THE INVENTION

Turfgrass, an agriculturally and economically important crop species, is used worldwide for lawns of buildings, roadsides, athletic and recreational fields providing numerous benefits including reducing soil erosion, trapping dust and pollutants, moderating temperature, providing safer playing grounds and beautifying the environment. There are more than 50 million acres of turfgrass and 16,000 golf courses in the US alone, and the turfgrass industry is a multibillion dollar business annually.

Turfgrasses are highly susceptible to a wide range of destructive fungal and bacteria pathogens, causing a great decrease in quality and safety. Chemical pesticides in a large amount are commonly and frequently applied in turfgrass disease management (Qu et al, 2008). However, frequent use and misuse of pesticides plus monoculture of high yielding varieties in agricultural ecosystems impose strong selection pressure on pathogens resulting in resistant pathogen strains and plant disease resistance collapse (Ma and Michailides, 2005). Chemical pesticides not only add a lot of operational costs but also arouse concerns over the hazards which they may pose to the environment. In the 1990s, the European Union, the US and other countries undertook regulatory changes in pesticide registration requirements, expecting a half reduction of existing ingredients (Montesinos, 2007). Under such circumstances, development of disease-resistant turfgrass using biotechnology approaches will not only improve turfgrass quality and greatly reduce turfgrass management cost, it will also significantly benefit the environment.

The food situation worldwide is becoming critical because of the vulnerability of modern agriculture to plant diseases. Pathogenic microorganisms, the leading cause of plant diseases and crop losses, require high amounts and continued use of chemical pesticides for disease control to satisfy the food needs (Rekha et al., 2006). However, the emergence of resistant pathogen strains, the limited spectrum of targets, and the negative long-term impact on human health and the environment have driven the search for new alternatives to currently used chemicals. Therefore, in agriculture, there is an urgent requirement to exploit products that present sustainable resistance to a broad range of pathogens and are safe for the host organisms, the consumers and the environment (Zasloff, 2002; Keymanesh, 2009).

Considering the above-mentioned concerns and expectations, antimicrobial peptides (AMPs) are suitable alternatives as substitutes to be used in various fields of agriculture. They are short sequence peptides with generally fewer than 50 amino acid residues, which have antimicrobial activity against microorganisms. They are a first line of defense in plants and animals which are ubiquitous in nature with high selectivity against target organisms, and resistance against them is much less observed compared with current antibiotics (Zasloff, 2002).

AMPs are diverse and can be subdivided into two major groups based on their electrostatic charges, which are the most important characteristic of AMPs (Vizioli and Salzet, 2002). The largest group of AMPs is that of cationic molecules, which are wildly distributed in plants and animals. The much smaller group of AMPs is that of non-cationic molecules including anionic peptides, aromatic peptides and peptides derived from oxygen-binding proteins. Compared with the first group, the non-cationic peptides are scarce and often the term “antimicrobial peptides (AMPs)” is used to refer only to cationic AMPs (Zasloff, 2002; Keymanesh, 2009).

On the basis of structural features, cationic AMPs can be subdivided into three classes: (1) linear peptides often adopting α-helical structures; (2) cysteine-rich open-ended peptides containing a single or several disulfide bridges; and (3) cyclopeptides forming a peptide ring (Montesinos, 2007). However, they also share certain common structural characteristics such as (1) amino acid composition in which cationic and hydrophobic residues are most abundant; (2) amphipathicity; and (3) a remarkable diversity of structures and conformations even including some non-conventional and extended structures (Vizioli and Salzet, 2002; Keymanesh, 2009). In fact, the second characteristic, amphipathicity, in many cases is membrane-induced, and this is an important property of cationic AMPs which can facilitate their interactions with microbial membranes (Zasloff, 2002). Some cationic AMPs are enriched in certain amino acids. For example, many cationic AMPs are rich in cysteines forming a single or several disulfide bridges (e.g., Ib-AMP4 from balsamine and penaeidins from shrimp), which makes their structures more compact and stable under various biochemical conditions such as protease degradation and so on. This group of AMPs is widespread in nature, including plants, animals, insects, and fungi, and exhibit a significant sequence and structure diversity (Vizioli and Salzet, 2002).

AMPs have been isolated from many organisms and act efficiently against pathogens without any damage to the host, because the structural differences between host and target cell membranes play an important role in the selective action of AMPs (Yount and Yeaman, 2005). The amino acid composition, amphipathicity, cationic charge and size of AMPs allow them to attach to and insert into bacterial membrane bilayers. After insertion into the membrane, antimicrobial peptides act by either disrupting the physical integrity of the bi-layer or by translocation across the membrane to act on internal targets. Several models describe these subsequent events, including the reorientation of peptide molecules perpendicular to the membrane to form either barrel-stave or toroidal channel, the breakdown of membrane integrity as a result of the swamping of membrane charge by a carpet of peptides at the interface, the detergent-like dissolution of patches of membrane and the formation of peptide-lipid aggregates within the bi-layer (Yount and Yeaman, 2005; Hancock, 2006).

A group of plant AMPs isolated from seeds of Impatiens balsamina (Ib-AMPs) (Tailor et al. 1997) have been found to be potent against bacterial and fungal infections but have no side effects on plant, animal and insect cells. Ib-AMPs, including Ib-AMP1, Ib-AMP2, Ib-AMP3 and Ib-AMP4, are part of a novel family of four highly homologous peptides. This family of peptides is the smallest of the antimicrobial peptides (20 amino acids) containing cysteines isolated from plants to date and has no sequence homology with previously identified AMPs (Tailor et al., 1997). Ib-AMPs are highly basic and contain four cysteine residues, which form two intramolecular disulfide bridges (Patel et al., 1998). The antifungal activity of the purified peptides was assessed on 13 fungal strains using a standard antifungal activity assay (Alternaria longipes, Botrytis cinerea, Cladosporium sphaerospermum, F. culmorum, Penicillium digitatum, T. viride, V. alboatrum, Colletotrichum gloeosporioides, Gloeodes pomigena, Gloeosporium solani, Nectria galligena, Phialophora malorum and Sclerotinia sclerotiorum), many of which are plant pathogens of significant importance to agriculture. In assay medium with low ionic strength, all four peptides showed similar levels of significant antifungal activity, and in the majority of the assays the IC₅₀ values were <10 μg/ml. However, when the assay medium was supplemented with 1 mM CaCl₂ and 50 mM KCl, the activity of Ib-AMP1, Ib-AMP2, and Ib-AMP3 was severely decreased. Only Ib-AMP4 maintained a significant inhibitory effect although its activity was also reduced. In studies with some fungi, such as N. crassa and F. culmorum, the Ib-AMPs produced a very distinct swelling and hyperbranching in the spore germination assay and also an inhibitory effect on the growth of germlings (Thevissen et al., 2005). Similarly, only Ib-AMP4 was able to maintain any significant inhibitory activity in the presence of cations with the activity of the other three peptides being dramatically reduced (Tailor et al., 1997).

In addition to their wide antifungal activity, the Ib-AMPs were also inhibitory to the growth of four Gram-positive bacteria (Bacillus subtilis, Micrococcus luteus, Staphylococcus aureus, Streptococcus faecalis) and to the growth of two Gram-negative Xanthomonas species. In these assays, the Ib-AMPs were more active on the four Gram-positive bacteria compared with another antibiotic peptide, magainin I (Tailor et al., 1997). Few of the other antifungal peptides isolated to date from plants show significant anti-bacteria activity (Osborn, 1995; Tailor et al., 1997).

The mode of action of the Ib-AMPs is presently unknown. Current data show that even at very high concentrations (500 μg/ml), the Ib-AMPs do not cause any visible cell lysis or membrane breakage on fungi (Tailor et al., 1997), and their activity for phospholipid disruption is very low compared with other α-helical amphiphatic, antimicrobial peptides (Lee et al., 1999). Confocal microscopy showed that biotinylated Ib-AMP 1 bound to the cell surface or penetrated into cell membranes (Lee et al., 1999). Taken together, these preliminary data suggest that the Ib-AMPs are not acting as ionophores but rather that they are inhibiting a distinct cellular process (Lee et al., 1999).

Besides plant derived AMPs, animal derived AMPs can also be considered for genetic engineering, for plant pathogens may have already evolved tolerance to plant derived AMPs, thus decreasing the effects of AMPs in vivo (Li et al, 2001). Moreover, a combination of plant derived and animal derived antimicrobial genes applied in genetic engineering allows for resistance to a broader range of bacteria and fungi.

Animal derived antimicrobial peptides have already been reported to confer resistance to plants (Li et al., 2001). An esculentin-1 encoding gene expressing a 46-residue AMP present in skin secretions of Rana esculenta, with the substitution Met-28Leu, was introduced into tobacco, and the transgenic plants indicated resistance against bacterial and fungal phytopathogens (Ponti et al, 2003). Expression of the mammalian antimicrobial peptide cecropin P1 in transgenic tobacco led to enhanced resistance to several phytopathogenic bacteria (Zakharchenko et al., 2005).

Penaeidins, a family of AMPs originally isolated from the haemocytes of penaeid shrimp Shrimp and other invertebrates lack the adaptive immune system which is characteristic of jawed vertebrates, thus relying exclusively on the innate immune system (Cuthbertson et al., 2004), in which penaeidin antimicrobial peptides are one of the key elements (Cuthbertson et al., 2006). Penaeidins make up a diverse peptide family with a unique two-domain structure including an unconstrained proline-rich N-terminal domain (PRD) and a cysteine-rich domain (CRD) with a stable α-helical structure (Cuthbertson et al., 2005). They are primarily directed against Gram-positive bacteria and fungi (Destoumieux et al., 1999) and are synthesized in granular haemocytes, released into the plasma upon microbial infection and localize to tissues, bound to cuticle surfaces (Destoumieux, 2000; Muñoz et al., 2002). The complexity inherent in the multi-domain structure of the peptide may contribute to its broad range of microbial targets (Yang et al., 2003; Destoumieux et al., 2000).

The penaeidin family is divided into four classes, designated 2, 3, 4 and 5 and each class displays a remarkable level of primary sequence diversity (Chen et al., 2004; Cuthbertson et al., 2006). Pen4-1 belongs to class four isoform one of the penaeidins isolated from Atlantic white shrimp (Litopenaeus setiferus). It contains six cysteine residues forming three disulfide bridges, and it is the shortest isoform in penaeidin family, with the length of 47 amino acids. It can inhibit multiple plant pathogenic fungal species, including B. cinera, P. crustosum, and F. oxysporum (Bachere et al., 2000). It is also effective against Gram-positive bacteria species including M. luteus and A. viriduans, and it is inhibitory against the Gram-negative bacterium, E. coli, at relatively high concentrations (Cuthbertson et al., 2006). Notably, Pen4-1 can fight against the multidrug-resistant fungal species Cryptococcus neoformans (Steroform A, Steroform B, Steroform C, Steroform D) and Candida spp (Candida lipolytica, Candida inconspicua, Candida krusei, Candida lusitaniae, Candida glabrata) (Cuthbertson et al., 2006). Compared with other penaeidins, penaeidin class 4 has shown a high level of effectiveness against fungi (Cuthbertson et al., 2006). Additionally, the unusual amino acid composition of PRD Pen4-1 may confer resistance to proteases (Cuthbertson et al., 2006).

Although the mechanism of action of penaeidins has not yet been revealed, recent work with other proline-rich AMPs indicates that internal cytosolic proteins can be the targets (Otvos, 2000; Cuthbertson et al., 2004). For example, the proline-rich AMP, apidaecin, originally isolated from honey bee, can not only penetrate the microbial membrane but also internalize itself, and then inhibit the function of heat-shock protein 70, which is an important molecular chaperone (Zasloff, 2002; Otvos 2000; Cuthbertson et al., 2004). A seemingly similar effect is observed for Litset Pen4-1 against filamentous fungi, and this effect implies a more complex mechanism than simple membrane disruption, i.e., targeting a specific microbial component conserved across phyla (Otvos, 2000; Cuthbertson et al., 2004, Cuthbertson et al., 2006).

The present invention addresses previous shortcomings in the art by providing methods and compositions employing combinations of plant and animal AMPs to enhance disease resistance in plants.

### SUMMARY OF THE INVENTION

In one aspect, the present invention provides a nucleic acid construct comprising: a) a nucleotide sequence encoding PEN4-1; b) a nucleotide sequence encoding Ib-AMP4; and c) a first promoter operably associated with the nucleotide sequence of (a).

In a further aspect, the present invention provides a nucleic acid construct comprising: a) a nucleotide sequence encoding PEN4-1; b) a nucleotide sequence encoding Ib-AMP4; and c) a promoter operably associated with the nucleotide sequence of (b).

Additional aspects of this invention provide the nucleic acid constructs of this invention, further comprising a termination sequence, a signal peptide sequence, a linker peptide, a selectable marker sequence, and any combination thereof.

The present invention also provides a nucleic acid construct of this invention, comprising in the following order from 5′ to 3′; a) a corn ubiquitin promoter; b) an AP24 signal peptide sequence; c) a nucleotide sequence encoding PEN4-1; d) an IbAMP propeptide; e) a nucleotide sequence encoding IbAMP-4; f) a first nos sequence; g) a rice ubiquitin promoter sequence; h) a bar coding sequence; and i) a second nos sequence.

Additionally provided herein is a nucleic acid construct of this invention, comprising in the following order from 5′ to 3′: a) a corn ubiquitin promoter; b) an AP24 signal peptide sequence; c) a nucleotide sequence encoding IbAMP4; d) an IbAMP propeptide; e) a nucleotide sequence encoding PEN4-1; f) a first nos sequence; g) a rice ubiquitin promoter sequence; h) a bar coding sequence; and i) a second nos sequence.

The present invention also provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding PEN4-1; i) a second nos sequence; j) a rice ubiquitin promoter sequence; k) a bar coding sequence; and 1) a third nos sequence.

In addition, the present invention provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding Ib-AMP-4; i) a second nos sequence; j) a rice ubiquitin promoter sequence; k) a bar coding sequence; and 1) a third nos sequence.

Also provided herein is a nucleic acid construct of this invention, comprising in the following order from 5′ to 3′: a) a maize ubiquitin promoter; b) a nucleotide sequence encoding PEN4-1; c) a first nos sequence; d) a CaMV 35S promoter sequence; e) a bar coding sequence; and f) a second nos sequence (pHL016; FIG. 10a).

An additional embodiment of this invention provides a nucleic acid construct, comprising, in the following order from 5′ to 3′: a) a maize ubiquitin promoter; b) an AP24 signal peptide sequence; c) a nucleotide sequence encoding PEN4-1; d) a first nos sequence; e) a CaMV 35S promoter sequence; f) a bar coding sequence; and g) a second nos sequence (pHL018; FIG. 10b).

Further aspects of this invention include a method of producing a transgenic plant having increased resistance to bacterial and/or fungal infection, comprising: a) transfoiming a cell of a plant with one or more nucleic acid constructs of this invention; and b) regenerating the transgenic plant from the transformed plant cell, wherein the plant has increased resistance to bacterial and or fungal infection as compared with a plant that is not transformed with said nucleic acid construct(s).

The present invention further provides a transgenic plant comprising one or more nucleic acid constructs of this invention, a transformed plant cell comprising one or more nucleic acid constructs of this invention and a vector comprising one or more nucleic acid of this invention

### BRIEF DESCRIPTION OF THE FIGURES

FIG. 1. Bacterial pathogen P. syringae pv. Tomato DC 3000 inoculation test in Arabidopsis: (A) Chimeric gene construct pSBUbi::AP24::Pen4, in which the corn ubiquitin promoter driving AP24::Pen4 fused gene is linked to a rice ubiquitin promoter driving herbicide resistance gene, bar. (B) Comparison of bacterial growth in the presence of plant extracts from wild type (WT) and transgenic (TG) plants. The plant extracts from transgenics harboring Ap24::Pen4-1 exhibited an inhibition effect on bacterial pathogen. Significant less bacterial growth than in the extracts from the wild-type control was observed. (C) The general procedure of the bacterial inoculation test in Arabidopsis is as follows: the same amount of Pst DC3000 bacterial suspension is pressure-infiltrated into the leaf intercellular space (C-1) of the WT and TG plants. The inoculated leaf samples were collected and grinded 2 days after inoculation (C-2). Then the samples were diluted and plated on the medium (C-3). The colony-forming units for each sample on the plates were counted two days after incubation at 28° C. (D) Statistical measurement of the number of the bacterial colonies on the plates with plant extracts from WT or TG. The inoculated bacteria in WT Arabidopsis exhibited significantly more growth than in the transgenic plants containing Ap24::Pen4-1.

FIGS. 2A-B. Transgenic turfgrass expressing Pen4-1 and AP24::Pen4-1 exhibited enhanced resistance on dollar spot disease (S. homeocarpa). The same amount of fungi was inoculated on both wild type (WT) and transgenic plants (TG). (B) shows the plants 10 days after inoculation. WT plants developed more severe symptoms than transgenic plants. (A) shows plants before inoculation.

FIGS. 3A-B. (A) pSBUbi::RNAi-Ubi::bar. (B) Transgenic Arabidopsis plants expressing an RNAi construction of creping bentgrass FLO/LFY homolog (left) and wild-type plants (right). Transgenic plants showed a delayed flowering.

FIG. 4. AMP gene constructions. #1, pSBUbi::AP24::Pen4-1-Ubi::bar. #2, pSBUbi::AP24::Ib-AMP4-Ubi::bar. #3, pSBUbi::AP24::Pen4-1::IbAMPpropeptide:Ib-AMP4-Ubi::bar. #4, pSBUbi::AP24::Ib-AMP4::IbAMPpropeptide::Pen4-1-Ubi::bar.

FIG. 5. #5, pSB35S::RNAi-Ubi::AP24::Pen4-1-Ubi::bar. #6, pSB35S::RNAi-Ubi::AP24::Ib-AMP4-Ubi::bar. #7, pSB35S::RNAi Ubi::AP24::Pen4::IbAMP propeptide::Ib-AMP4-Ubi::bar. #8, pSB35S::RNAi Ubi::AP24::Ib-AMP4::IbAMP propeptide::Pen4-1-Ubi::bar.

FIG. 6. #2 (pSBUbi::AP24::Ib-AMP4-Ubi::bar) and #5 (pSB35S::RNAi-Ubi::AP24::Pen4-1-Ubi::bar) will be co-transformed.

FIG. 7. #1 (pSBUbi::AP24::Pen-1-Ubi::bar) and #6 (pSB35S::RNAi-Ubi::AP24::Ib-AMP4-Ubi::bar.) will be co-transformed.

FIG. 8. Agrobacterium-mediated plant transformation and flowchart of the turfgrass tissue culture: (1) selection of the transformed turfgrass callus; (2-3) regeneration of the transformed turfgrass callus on shooting medium and then on rooting medium; (4) putative transformed plants transferred to soil; (5) plants are treated with herbicide; non-transformed turfgrass died after herbicide treatment, whereas transformed turfgrass maintained vigorous growth.

FIG. 9. Amino acid sequences and modified DNA sequences of Pen4-1 gene.

FIGS. 10A-D. Generation and molecular analysis of the transgenic pHL016 (Pen4-1). (a) Schematic diagram of the Pen4-1-expression chimeric gene construct, pSBbarB/Ubi-Pen4-1. Pen4-1 gene is under the control of the maize ubiquitin promoter and linked to the herbicide resistance gene, bar, driven by a CaMV 35S promoter. (b) Schematic diagram of the AP24::Pen4-1-expression chimeric gene construct, pSBbarB/Ubi-AP24::Pen4-1. (c) Example of Southern blot analysis of Pen4-1-expressing transgenics. Twenty μg of the genomic DNA extracted from young leaves and digested with BamHI that cuts once within the T-DNA region was probed by a 440 by ³²P-labelled bar gene fragment. Hybridization signals revealed were indication of copy numbers of transgene insertion. Lanes 1-15 were DNAs from representative transgenic creeping bentgrass plants. The negative control (WT) was BamHI-digested genomic DNA from a non-transformed wild-type plant. (d) Example of Northern blot analysis of Pen4-1-expressing transgenics. Lanes 1-8 were total RNA from the same representative transgenic creeping bentgrass plants used for Southern analysis in (b). Twenty μg of the total RNA extracted from young leaves and probed with a 1.5 kb ³²P-labelled Pen4-1 gene fragment. The negative control (WT) was total RNA from a non-transformed wild-type plant.

FIGS. 11A-B. In vitro plant leaf inoculation test with R. solani. (a) Representative leaves of transgenic plants (on the right) and wild type plants as control (on the left) on 14 days post inoculation with R. solani. (b) Statistical analysis of R. solani inoculation test on the transgenic lines pHL016-4 (Pen4-1), pHL016-8 (Pen4-1), pHL018-1 (AP24::Pen4-1) and pHL018-3 (AP24::Pen4-1). Lesion length data of 2 DPI, 8 DPI and 14 DPI were documented and on 14DPI, transgenic plants showed significant resistance to R. solani in comparison to wild type plants. Error bars represent standard deviation. Asterisks (*) indicate a significant difference between transgenic plants and wild-type controls at P<0.05 by ANOVA using minitab 16.

FIGS. 12A-C. In vivo direct plant inoculation bioassays with first dose of R. solani. (a) Plants before inoculation were shown in the upper rows, and the lower rows represent the plants 14 days post inoculation. Wild type plants (left) exhibited more sever symptoms than transgenic creeping bentgrass lines pHL016-4 (Pen4-1), pHL018-1 (AP24::Pen4-1), pHL018-3 (AP24::Pen4-1), pHL018-5 (AP24::Pen4-1) (right) two weeks after inoculation. (b) A closer look of the different lesion size of WT and TG. (c) Plant lesion diameters of wild type plants as control (WT), transgenic lines pHL016-4 (Pen4-1), pHL018-1 (AP24::Pen4-1), pHL018-3 (AP24::Pen4-1) and pHL018-5 (AP24::Pen4-1) (TG) on 14 days post inoculation with R. solani.

FIGS. 13A-C. In vivo direct plant inoculation bioassays with second dose of R. solani. (a) Wild type plants (left) exhibited more sever symptoms than transgenic creeping bentgrass lines pHL016-4 (Pen4-1), pHL018-1 (AP24::Pen4-1) and pHL018-3 (AP24::Pen4-1) (right) two weeks after second inoculation of R. solani. (b) A closer look of the different lesion size of WT and TG. (c) Plant disease ratings of wild type plants as control (WT), transgenic lines pHL016-4 (Pen4-1), pHL018-1 (AP24::Pen4-1) and pHL018-3 (AP24::Pen4-1) on 14 days post inoculation with second dose of R. solani.

FIGS. 14A-B. In vitro plant leaf inoculation test with S. homoeocarpa (a) Representative leaves of wild type plants (WT) and transgenic plants (TG) on 7 days post inoculation with S. homoeocarpa. (b) Statistical analysis of S. homoeocarpa inoculation test on the transgenic lines pHL016-4 (Pen4-1), pHL016-8 (Pen4-1), pHL018-1 (AP24::Pen4-1) and pHL018-3 (AP24::Pen4-1). (b) Lesion length data of 2 DPI, 4 DPI and 7 DPI were documented, and on 7DPI transgenic plants showed significant resistance to S. homoeocarpa in comparison to wild type plants.

FIGS. 15A-B. In vivo direct plant inoculation bioassays (a) wild type (left) and transgenic creeping bentgrass line pHL016-4 (Pen4-1) (middle) and pHL018-1 (AP24::Pen4-1) (right) 9 days after inoculation with S. homoeocarpa. Infected plants are shown in the upper rows respectively, and the lower rows represent the plants which have been not infected. (b) Plant disease ratings of non-transformed wild type plants as control (WT), transgenic lines pHL016-14 (Pen4-1) and pHL018-3 (AP24::Pen4-1) (TG) on 3 days, 5 days, 7 days, 9 days post inoculation with S. homoeocarpa and 21 days post recovery (DPR).

FIG. 16. Plasmid pHL17 (pGM-T-AP24-PEN4). Pen4-1 gene was fused to the signal peptide sequence of AP24 gene.

### DETAILED DESCRIPTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

The present invention is based on the unexpected discovery that the introduction into a plant of one or more of the nucleic acid constructs of this invention, which comprise nucleotide sequence(s) encoding one or more antimicrobial peptides, results in the production of a transgenic plant having increased or enhanced resistance to various plant pathogens.

Thus, in one embodiment, the present invention provides a nucleic acid construct comprising one or more (e.g., 2, 3, 4, 5, 6, 7, 8, 9, 10, etc) nucleotide sequences encoding an antimicrobial peptide and operably associated with a promoter. In various embodiments, the antimicrobial peptide can be of plant origin, animal origin or microbial origin. The nucleic acid construct can comprise, consist essentially of and/or consist of a single nucleotide sequence encoding an AMP as well as multiple nucleotide sequences encoding an AMP. The AMPs can be combined on a single construct in any combination (e.g., from plant, animal and/or microbial origin, in any order and in any combination of multiples). For example, a nucleic acid construct of this invention can comprise a plant AMP and an animal AMP. In another example a nucleic acid construct of this invention can comprise two different plant AMPs and two different animal AMPs, in any combination and in any order. Thus, in one embodiment, a nucleic acid construct of this invention comprises a nucleotide sequence encoding an animal AMP and a nucleotide sequence encoding a plant AMP, and a promoter operably associated with the animal AMP, the plant AMP or both.

Nonlimiting examples of an antimicrobial peptide (AMP) of plant origin include AMPs from Impatiens balsamina (e.g., Ib-AMP1, Ib-AMP2, Ib-AMP3, Ib-AMP4); AMP from alfalfa (Medicago sativa) (e.g., alfAFP; Gao et al. 2000); AMP from morning glory (Pharbitis nil) (e.g., Pn-AMP2; Koo et al. 2002); AMP from Mirabilis jalapa (e.g., Mj-AMP1; Schaefer et al. 2005); AMP from Macadamia integrifolia (e.g., MiAMPl; Kazan et al. 2002); and AMP for pea (e.g., pea defensin; Wang et al. 1999).

Nonlimiting examples of AMPs of animal origin include AMPs from shrimp (e.g., penaeidins such as Litvan PEN3-1, Litvan PEN3-2, Litvan PEN3-3, Litvan PEN3-4, Litvan. PEN3-5, Litvan PEN3-6, Litvan PEN3-7, Litvan PEN3-8, Litvan PEN3-9, Litvan PEN3-10, Litvan PEN3-11, Litsty PEN3-1, Litsty PEN3-2, Litset PEN3-1, Litset PEN3-2, Litset PEN3-3, Litset PEN3-4, Penmon PEN3-1, Penmon PEN3-2, Penmon PEN3-3, Fenchi PEN3-1, Pensem PEN3-1, Litset PEN4-1, Litvan PEN4-1, Litvan PEN4-2, Litsch PEN4-1, Litvan PEN2-1, Litvan PEN2-2, Litvan PEN2-3, Litsty PEN2-1, Litset PEN2-1, Litsch PEN2-1, Litsch PEN2-2, Farpau PEN2-1 and Farpau PEN2-2; Gueguen et al. “PenBase, the shrimp antimicrobial peptide penaeidin database: Sequence-based classification and recommended nomenclature” Dev Comp Immunol. 30(3):283-288 (2006)); AMPs from honey bee (e.g., apidaecin; Zasloff 2002, Otvos 2000, Cuthbertson et al. 2004); AMPs from Xenopus laevis (e.g., magainin I, magainin II; Tailor et al. 1997); AMPs from Hyalophora cecropin (giant silkmoth) and Bombyx mori (domesticated silkmoth) [e.g., cecropin B (Sharma et al. 2000; Chiou et al. 2002); cecropin A (Coca et al. 2006)]; AMPs from cow (Bos taurus) (e.g., lactoferricin; Zhang et al.), AMPs from Rana esculenta (e.g., esculentin-1 (Ponti et al. 2003) and AMPs from Tachypleus tridentatus (e.g., tachyplesin; Lu 2003).

Nonlimiting examples of AMPs of microbial origin include AMPs from Streptomyces cacaoi (e.g., polyoxin; Reuveni et al. 2000; Arakawa 2003) and AMPs from Lactococcus lactis (e.g., nisin; Delves 2005).

In certain embodiments the present invention provides a nucleic acid construct comprising: a) a nucleotide sequence encoding PEN4-1; b) a nucleotide sequence encoding Ib-AMP4; and c) a first promoter operably associated with the nucleotide sequence of (a).

Also provided herein is a nucleic acid construct comprising: a) a nucleotide sequence encoding PEN4-1; b) a nucleotide sequence encoding Ib-AMP4; and c) a promoter operably associated with the nucleotide sequence of (b). In particular embodiments of these nucleic acid constructs, the promoter can be a corn ubiquitin promoter.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “promoter” or “plant promoter” of this invention is a promoter capable of initiating transcription in plant cells. Such promoters include those that drive expression of a nucleotide sequence constitutively, those that drive expression when induced, and those that drive expression in a tissue- or developmentally-specific manner, as these various types of promoters are known in the art.

Thus, for example, in some embodiments of the invention, a constitutive promoter can be used to drive the expression of a transgene of this invention in a plant cell. A constitutive promoter is an unregulated promoter that allows for continual transcription of its associated gene or coding sequence. Thus, constitutive promoters are generally active under most environmental conditions, in most or all cell types and in most or all states of development or cell differentiation.

Any constitutive promoter functional in a plant can be utilized in the instant invention. Exemplary constitutive promoters include, but are not limited to, the promoters from plant viruses including, but not limited to, the 35S promoter from CaMV (Odell et al., Nature 313: 810 (1985)); figwort mosaic virus (FMV) 35S promoter (P-FMV35S, U.S. Pat. Nos. 6,051,753 and 6,018,100); the enhanced CaMV35S promoter (e35S); the 1′- or 2′-promoter derived from T-DNA of Agrobacterium tumefaciens; the nopaline synthase (NOS) and/or octopine synthase (OCS) promoters, which are carried on tumor-inducing plasmids of Agrobacterium tumefaciens (Ebert et al., Proc. Natl. Acad. Sci. (U.S.A.), 84:5745 5749, 1987); actin promoters including, but not limited to, rice actin (McElroy et al., Plant Cell 2: 163 (1990); U.S. Pat. No. 5,641,876); histone promoters; tubulin promoters; ubiquitin and polyubiquitin promoters, including a corn ubiquitin promoter or a rice ubiquitin promoter ((Sun and Callis, Plant J., 11(5):1017-1027 (1997)); Christensen et al., Plant Mol. Biol 12: 619 (1989) and Christensen et al., Plant Mol. Biol. 18: 675 (1992)); pEMU (Last et al., Theor. Appl. Genet. 81: 581 (1991)); the mannopine synthase promoter (MAS) (Velten et al., EMBO J. 3: 2723(1984)); maize H3 histone (Lepelit et al., Mol. Gen. Genet. 231: 276 (1992) and Atanassova et al., Plant Journal 2: 291 (1992)); the ALS promoter, a Xbal/Ncol fragment 5′ to the Brassica napus ALS3 structural gene (or a nucleotide sequence that has substantial sequence similarity to said Xbal/Ncol fragment); ACT11 from Arabidopsis (Huang et al., Plant Mol. Biol. 33:125-139 (1996)); Cat3 from Arabidopsis (GenBank No. U43147, Zhong et al., Mol. Gen. Genet. 251:196-203 (1996)); GPc1 from maize (GenBank No. X15596, Martinez et al., J. Mol. Biol. 208:551-565 (1989)); and Gpc2 from maize (GenBank No. U45855, Manjunath et al., Plant Mol. Biol. 33:97-112 (1997)), including any combination thereof.

In some embodiments of the present invention, an inducible promoter can be used to drive the expression of a transgene. Inducible promoters activate or initiate expression only after exposure to, or contact with, an inducing agent. Inducing agents include, but are not limited to, various environmental conditions (e.g., pH, temperature), proteins and chemicals. Examples of environmental conditions that can affect transcription by inducible promoters include pathogen attack, anaerobic conditions, extreme temperature and/or the presence of light. Examples of chemical inducing agents include, but are not limited to, herbicides, antibiotics, ethanol, plant hormones and steroids. Any inducible promoter that is functional in a plant can be used in the instant invention (see, Ward et al., (1993) Plant Mol. Biol. 22: 361 (1993)). Exemplary inducible promoters include, but are not limited to, promoters from the ACEI system, which respond to copper (Melt et al., PNAS 90: 4567 (1993)); the ln2 gene from maize, which responds to benzenesulfonamide herbicide safeners (Hershey et al., (1991) Mol. Gen. Genetics 227: 229 (1991) and Gatz et al., Mol. Gen. Genetics 243: 32 (1994)); a heat shock promoter, including, but not limited to, the soybean heat shock promoters Gmhsp 17.5-E, Gmhsp 17.2-E and Gmhsp 17.6-L and those described in U.S. Pat. No. 5,447,858; the Tet repressor from Tn10 (Gatz et al., Mol. Gen. Genet. 227: 229 (1991)) and the light-inducible promoter from the small subunit of ribulose bisphosphate carboxylase (ssRUBISCO), including any combination thereof. Other examples of inducible promoters include, but are not limited to, those described by Moore et al. (Plant J. 45:651-683 (2006)). Additionally, some inducible promoters respond to an inducing agent to which plants do not normally respond. An example of such an inducible promoter is the inducible promoter from a steroid hormone gene, the transcriptional activity of which is induced by a glucocorticosteroid hormone (Schena et al., Proc. Natl. Acad. Sci. U.S.A. 88: 421 (1991)).

In further embodiments of the present invention, a tissue-specific promoter can be used to drive the expression of a transgene in a particular tissue in the transgenic plant. Tissue-specific promoters drive expression of a nucleic acid only in certain tissues or cell types, e.g., in the case of plants, in the leaves, stems, flowers and their various parts, roots, fruits and/or seeds, etc. Thus, plants transformed with a nucleic acid of interest operably linked to a tissue-specific promoter produce the product encoded by the transgene exclusively, or preferentially, in a specific tissue or cell type.

Any plant tissue-specific promoter can be utilized in the instant invention. Exemplary tissue-specific promoters include, but are not limited to, a root-specific promoter, such as that from the phaseolin gene (Murai et al., Science 23: 476 (1983) and Sengupta-Gopalan et al., Proc. Natl. Acad. Sci. USA 82: 3320 (1985)); a leaf-specific and light-induced promoter such as that from cab or rubisco (Simpson et al. EMBO J. 4: 2723 (1985) and Timko et al., Nature 318: 579 (1985)); the fruit-specific E8 promoter from tomato (Lincoln et al. Proc. Nat'l. Acad. Sci. USA 84: 2793-2797 (1988); Deikman et al. EMBO J. 7; 3315-3320 (1988); Deikman et al. Plant Physiol. 100: 2013-2017 (1992); seed-specific promoters of, for example, Arabidopsis thaliana (Krebbers et al. (1988) Plant Physiol. 87:859); an anther-specific promoter such as that from LAT52 (Twell et al. Mol. Gen. Genet. 217: 240 (1989)) or European Patent Application No 344029, and those described by Xu et al. (Plant Cell Rep. 25:231-240 (2006)) and Gomez et al. (Planta 219:967-981 (2004)); a pollen-specific promoter such as that from Zm13 (Guerrero et al., Mol. Gen. Genet. 224: 161 (1993)), and those described by Yamaji et al. (Plant Cell Rep. 25:749-57 (2006)) and Okada et al. (Plant Cell Physiol. 46:749-802 (2005)); a pith-specific promoter, such as the promoter isolated from a plant TrpA gene as described in International PCT Publication No. WO93/07278; and a microspore-specific promoter such as that from apg (Twell et al. Sex. Plant Reprod. 6: 217 (1993)). Exemplary green tissue-specific promoters include the maize phosphoenol pyruvate carboxylase (PEPC) promoter, small subunit ribulose bis-carboxylase promoters (ssRUBISCO) and the chlorophyll a/b binding protein promoters, including any combination thereof.

A promoter of the present invention can also be developmentally specific in that it drives expression during a particular “developmental phase” of the plant. Thus, such a promoter is capable of directing selective expression of a nucleotide sequence of interest at a particular period or phase in the life of a plant (e.g., seed formation), compared to the relative absence of expression of the same nucleotide sequence of interest in a different phase (e.g. seed germination). For example, in plants, seed-specific promoters are typically active during the development of seeds and germination promoters are typically active during germination of the seeds. Any developmentally-specific promoter capable of functioning in a plant can be used in the present invention.

The nucleic acid construct can further comprise a termination sequence. Nonlimiting examples of a termination sequence of this invention include the nopaline synthase (nos) sequence, gene 7 poly(A) signal, and CaMV 35S gene poly(A) signal.

The nucleic acid construct of this invention can further comprise a signal peptide sequence. Nonlimiting examples of a signal peptide sequence include the signal sequence of the tobacco AP24 protein (Coca et al. 2004); the signal peptide of divergicin A (Worobo et al. 1995); the proteinase inhibitor II signal peptide (Herbers et al. 1995); and the signal peptide from a Coix prolamin (Leite et al. 2000, Ottoboni et al. (1993), including any combination thereof.

The nucleic acid construct of this invention can further comprise a linker peptide. Nonlimiting examples of a linker peptide of this invention include the IbAMP propeptide (Francois et al. 2002, Sabelle et al. 2002); the 2A sequence of foot and mouth disease virus (Ma et al. 2002); and a serine rich peptide linker [e.g., Ser, Ser, Ser, Ser, Gly)_{y} where y≧1 (U.S. Pat. No. 5,525,491), including any combination thereof.

The nucleic acid constructs of the present invention can further comprise a nucleotide sequence encoding a selectable marker, operably linked to a regulatory element (a promoter, for example) that allows transformed cells in which the expression product of the selectable marker sequence is produced, to be recovered by either negative selection, i.e., inhibiting growth of cells that do not contain the selectable marker, or positive selection, i.e., screening for the product encoded by the selectable marker coding sequence. For example, in one embodiment the nucleic acid construct can comprise a phosphinothricin acetyltransferase (bar) coding sequence operably associated with a rice ubiquitin promoter sequence.

Many commonly used selectable marker coding sequences for plant transformation are well known in the transformation art, and include, for example, nucleotide sequences that code for enzymes that metabolically detoxify a selective chemical agent which may be an antibiotic or a herbicide, and/or nucleotide sequences that encode an altered target which is insensitive to the inhibitor (See e.g., Aragão et al., Braz. J. Plant Physiol. 14: 1-10 (2002)). Any nucleotide sequence encoding a selectable marker that can be expressed in a plant is useful in the present invention.

One commonly used selectable marker coding sequence for plant transformation is the nucleotide sequence encoding neomycin phosphotransferase II (npfII), isolated from transposon Tn5, which when placed under the control of plant regulatory signals confers resistance to kanamycin (Fraley et al., Proc. Natl. Acad. Sci. U.S.A., 80: 4803 (1983)). Another commonly used selectable marker coding sequence encodes hygromycin phosphotransferase, which confers resistance to the antibiotic hygromycin (Vanden Elzen et al., Plant Mol. Biol., 5: 299 (1985)).

Some selectable marker coding sequences confer resistance to herbicides. Herbicide resistance sequences generally encode a modified target protein insensitive to the herbicide or an enzyme that degrades or detoxifies the herbicide in the plant before it can act (DeB lock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990)). For example, resistance to glyphosate or sulfonylurea herbicides has been obtained using marker sequences coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial nucleotide sequences encoding phosphinothricin acetyltransferase, a nitrilase, or a 2,4-dichlorophenoxyacetate monooxygenase, which detoxify the respective herbicides.

Other selectable marker coding sequences for plant transformation are not of bacterial origin. These coding sequences include, for example, mouse dihydrofolate reductase, plant 5-eno/pyruvylshikimate-3-phosphate synthase and plant acetolactate synthase (Eichholtz et al., Somatic Cell Mol. Genet. 13: 67 (1987); Shah et al., Science 233: 478 (1986); Charest et al., Plant Cell Rep. 8: 643 (1990)).

Another class of marker coding sequences for plant transformation requires screening of presumptively transformed plant cells rather than direct genetic selection of transformed cells for resistance to a toxic substance such as an antibiotic. These coding sequences are particularly useful to quantify or visualize the spatial pattern of expression of a nucleotide sequence in specific tissues and are frequently referred to as reporter nucleotide sequences because they can be fused to a gene or gene regulatory sequence for the investigation of gene expression. Commonly used nucleotide sequences for screening presumptively transformed cells include, but are not limited to, those encoding β-glucuronidase (GUS), β-galactosidase, luciferase and chloramphenicol acetyltransferase (Jefferson Plant Mol. Biol. Rep. 5:387 (1987); Teeri et al. EMBO J 8:343 (1989); Koncz et al. Proc. Natl. Acad. Sci. U.S.A. 84:131 (1987); De Block et al. EMBO J. 3:1681 (1984)).

Some in vivo methods for detecting GUS activity that do not require destruction of plant tissue are available (e.g., Molecular Probes Publication 2908, Imagene Green™, p. 1-4 (1993) and Naleway et al., J. Cell Biol. 115:15 (1991)). In addition, a nucleotide sequence encoding green fluorescent protein (GFP) has been utilized as a marker for expression in prokaryotic and eukaryotic cells (Chalfie et al., Science 263:802 (1994)). GFP and mutants of GFP may be used as screenable markers. Similar to GFP, red fluorescent protein (DsRed2) has also been used as a selectable marker in plants (Nishizawa et al., Plant Cell Reports 25 (12): 1355-1361 (2006)). In addition, reef coral proteins have been used as selectable markers in plants (Wenck et al. Plant Cell Reports 22(4):244-251 (2003)).

For purposes of the present invention, selectable marker coding sequences can also include, but are not limited to, nucleotide sequences encoding: neomycin phosphotransferase I and II (Southern et al., J. Mol. Appl. Gen. 1:327 (1982)); Fraley et al., CRC Critical Reviews in Plant Science 4:1 (1986)); cyanamide hydratase (Maier-Greiner et al., Proc. Natl. Acad. Sci. USA 88:4250 (1991)); aspartate kinase; dihydrodipicolinate synthase (Perl et al., BioTechnology 11, 715 (1993)); bar gene (Toki et al., Plant Physiol. 100:1503 (1992); Meagher et al., Crop Sci. 36:1367 (1996)); tryptophane decarboxylase (Goddijn et al., Plant Mol. Biol. 22:907 (1993)); hygromycin phosphotransferase (HPT or HYG; Shimizu et al., Mol. Cell. Biol. 6:1074 (1986); Waldron et al., Plant Mol. Biol. 5:103 (1985); Zhijian et al., Plant Science 108:219 (1995)); dihydrofolate reductase (DHFR; Kwok et al., Proc. Natl. Acad. Sci. USA 83:4552 (1986)); phosphinothricin acetyltransferase (DeBlock et al., EMBO J. 6:2513 (1987)); 2,2-dichloropropionic acid dehalogenase (Buchanan-Wollatron et al., J. Cell. Biochem. 13D:330 (1989)); acetohydroxyacid synthase (U.S. Pat. No. 4,761,373 to Anderson et al.; Haughn et al., Mol. Gen. Genet. 221:266 (1988)); 5-enolpyruvyl-shikimate-phosphate synthase (aroA; Comai et al., Nature 317:741 (1985)); haloarylnitrilase (PCT Publication No. WO 87/04181 to Stalker et al.); acetyl-coenzyme A carboxylase (Parker et al., Plant Physiol. 92:1220 (1990)); dihydropteroate synthase (sulI; Guerineau et al., Plant Mol. Biol. 15:127 (1990)); and 32 kDa photosystem II polypeptide (psbA; Hirschberg et al., Science 222:1346 (1983)).

Also included are nucleotide sequences that encode polypeptides that confer resistance to: gentamicin (Miki et al., J. Biotechnol. 107:193-232 (2004)); chloramphenicol (Herrera-Estrella et al., EMBO J. 2:987 (1983)); methotrexate (Herrera-Estrella et al., Nature 303:209 (1983); Meijer et al., Plant Mol. Biol. 16:807 (1991)); Meijer et al., Plant Mol. Bio. 16:807 (1991)); streptomycin (Jones et al., Mol. Gen. Genet. 210:86 (1987)); spectinomycin (Bretagne-Sagnard et al., Transgenic Res. 5:131 (1996)); bleomycin (Hille et al., Plant Mol. Biol. 7, 171 (1986)); sulfonamide (Guerineau et al., Plant Mol. Bio. 15:127 (1990); bromoxynil (Stalker et al., Science 242:419 (1988)); 2,4-D (Streber et al., Bio/Technology 7, 811 (1989)); phosphinothricin (DeBlock et al., EMBO J. 6:2513 (1987)); and/or spectinomycin (Bretagne-Sagnard and Chupeau, Transgenic Research 5:131 (1996)).

The product of the bar gene confers herbicide resistance to glufosinate-type herbicides, such as phosphinothricin (PPT) or bialaphos, and the like. As noted above, other selectable markers that could be used in the nucleic acid constructs of the present invention include, but are not limited to, the pat gene or coding sequence, the expression of which also confers resistance to bialaphos and phosphinothricin resistance, the ALS gene or coding sequence for imidazolinone resistance, the HPH or HYG gene or coding sequence for hygromycin resistance (Coca et al. 2004), the EPSP synthase gene or coding sequence for glyphosate resistance, the Hml gene or coding sequence for resistance to the Hc-toxin, a coding sequence for streptomycin phosphotransferase resistance (Mazodier et al.) and/or other selective agents used routinely and known to one of ordinary skill in the art. See generally, Yarranton, Curr. Opin. Biotech. 3:506 (1992); Chistopherson et al., Proc. Natl. Acad. Sci. USA 89:6314 (1992); Yao et al., Cell 71:63 (1992); Reznikoff, Mol. Microbial. 6:2419 (1992); Barkley et al., The Operon 177-220 (1980); Hu et al., Cell 48:555 (1987); Brown et al., Cell 49:603 (1987); Figge et al., Cell 52:713 (1988); Deuschle et al., Proc. Natl. Acad. Sci. USA 86:400 (1989); Fuerst et al., Proc. Natl. Acad. Sci. USA 86:2549 (1989); Deuschle et al., Science 248:480 (1990); Labow et al., Mol. Cell. Biol. 10:3343 (1990); Zambretti et al., Proc. Natl. Acad. Sci. USA 89:3952 (1992); Baim et al., Proc. Natl. Acad. Sci. USA 88:5072 (1991); Wyborski et al., Nuc. Acids Res. 19:4647 (1991); Hillenand-Wissman, Topics in Mol. And Struc. Biol. 10:143 (1989); Degenkolb et al., Antimicrob. Agents Chemother. 35:1591 (1991); Kleinschnidt et al., Biochemistry 27:1094 (1988); Gatz et al., Plant J. 2:397 (1992); Gossen et al., Proc. Natl. Acad. Sci. USA 89:5547 (1992); Oliva et al., Antimicrob. Agents Chemother. 36:913 (1992); Hlavka et al., Handbook of Experimental Pharmacology 78 (1985); and Gill et al., Nature 334:721 (1988). A review of approximately 50 marker genes in transgenic plants is provided in Miki et al. (2003), the entire contents of which are incorporated by reference herein.

Additionally, for purposes of the present invention, selectable markers include nucleotide sequence(s) conferring environmental or artificial stress resistance or tolerance including, but not limited to, a nucleotide sequence conferring high glucose tolerance, a nucleotide sequence conferring low phosphate tolerance, a nucleotide sequence conferring mannose tolerance, and/or a nucleotide sequence conferring drought tolerance, salt tolerance or cold tolerance. Examples of nucleotide sequences that confer environmental or artificial stress resistance or tolerance include, but are not limited to, a nucleotide sequence encoding trehalose phosphate synthase, a nucleotide sequence encoding phosphomannose isomerase (Negrotto et al., Plant Cell Reports 19(8):798-803 (2003)), a nucleotide sequence encoding the Arabidopsis vacuolar H⁺-pyrophosphatase gene, AVP1, a nucleotide sequence conferring aldehyde resistance (U.S. Pat. No. 5,633,153), a nucleotide sequence conferring cyanamide resistance (Weeks et al., Crop Sci 40:1749-1754 (2000)) and those described by luchi et al. (Plant J. 27(4):325-332 (2001)); Umezawa et al. (Curr Opin Biotechnol. 17(2):113-22 (2006)); U.S. Pat. No. 5,837,545; Oraby et al. (Crop Sci. 45:2218-2227 (2005)) and Shi et al. (Proc. Natl. Acad. Sci. 97:6896-6901 (2000)).

The above list of selectable marker genes and coding sequences is not meant to be limiting as any selectable marker coding sequence now known or later identified can be used in the present invention. Also, a selectable marker of this invention can be used in any combination with any other selectable marker.

In some embodiments of this invention, the nucleic acid construct of this invention can comprise gene elements to control gene flow in the environment in which a transgenic plant of this invention could be placed. Examples of such elements are described in International Publication No. WO 2009/011863, the disclosures of which are incorporated by reference herein.

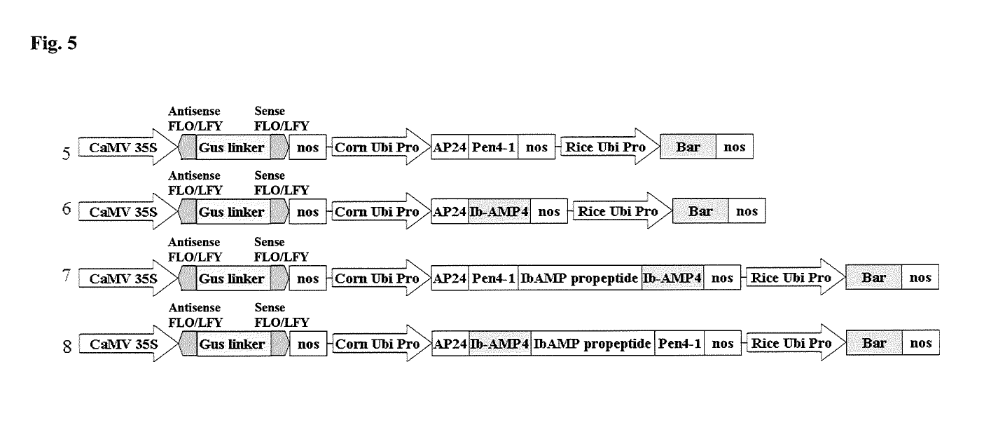

In some embodiments, the nucleic acid construct of this invention can comprise elements to impart sterility to the transgenic plant into which the nucleic acid construct is introduced in order to control movement of the transgene(s) of this invention in the environment. As one example, RNAi technology can be used to turn off the expression of certain endogenous genes, resulting in a plant that maintains vegetative growth during its whole life cycle. In particular examples the LFY gene of Arabidopsis and the FLO/LFY homolog in creeping bentgrass can be targeted by interfering RNA molecules according to well known techniques to inhibit expression of these genes in the transgenic plant and producing sterility in the transgenic plant. Examples of nucleic acid constructs of this invention are shown in FIGS. 5, 6 and 7.

Elements that can impart sterility to the transgenic plant include, but are not limited to, nucleotide sequences, or fragments thereof, that modulate the reproductive transition from a vegetative meristem or flower promotion gene or coding sequence, or flower repressor gene or coding sequence. Three growth phases are generally observed in the life cycle of a flowering plant: vegetative, inflorescence and floral. The switch from vegetative to reproductive or floral growth requires a change in the developmental program of the descendents of the stem cells in the shoot apical meristem. In the vegetative phase, the shoot apical meristem generates leaves that provide resources necessary to produce fertile offspring. Upon receiving the appropriate environmental and developmental signals, the plant switches to floral (reproductive) growth and the shoot apical meristem enters the inflorescence phase, giving rise to an inflorescence with flower primordia. During this phase, the fate of the shoot apical meristem and the secondary shoots that arise in the axils of the leaves is determined by a set of meristem identity genes, some of which prevent and some of which promote the development of floral meristems. Once established, the plant enters the late inflorescence phase where the floral organs are produced. Two basic types of inflorescences have been identified in plants: determinate and indeterminate. In a species producing a determinate inflorescence, the shoot apical meristem eventually produces floral organs and the production of meristems is terminated with a flower. In those species producing an indeterminate inflorescence, the shoot apical meristem is not converted to a floral identity and therefore only produces floral meristems from its periphery, resulting in a continuous growth pattern.

In dicots, after the transition from vegetative to reproductive development, floral meristems are initiated by the action of a set of genes called floral meristem identity genes. FLORICAULA (flo) of Antirrhinum and its Arabidopsis counterpart, LEAFY (lfy), are floral meristem identity genes that participate in the reproductive transition to establish floral fate. In strong flo and lfy mutant plants, flowers are transformed into inflorescence shoots (Coen et al., Cell 63:1311-1322 (1990); Weigel et al. Cell 69:843-859, (1992)), indicating that flo and lfy are exemplary flower-promotion genes.

In monocots, FLO/LFY homologs have been identified in several species, such as rice (Kyozuka et al., Proc. Natl. Acad. Sci. 95:1979-1982 (1998)); Lolium temulentum, maize, and ryegrass (Lolium perenne). The FLO/LFY homologs from different species have high amino acid sequence homology and are well conserved in the C-terminal regions (Kyozuka et al., Proc. Natl. Acad. Sci. 95:1979-1982 (1998); Bomblies et al., Development 130:2385-2395 (2003)).

In addition to flo/lfy genes or coding sequences, other examples of flower promotion genes or coding sequences include, but are not limited to, APETALA1 (Accession no. NM105581)/SQUAMOSA (apl/squa) in Arabidopsis and Antirrhinum, CAULIFLOWER (cal, Accession no. AY174609), FRUITFUL (ful, Accession no. AY173056), FLOWERING LOCUS T (Accession no. AB027505), and SUPPRESSOR OF OVEREXPRESSION OF CONSTANS1 (sod) in Arabidopsis (Samach et al., Science 288:1613-1616 (2000); Simpson and Dean, Science 296:285-289 (2002)); Zik et al., Annu. Rev. Cell Dev. Biol. 19:119-140 (2003)).

Additional non-limiting examples of flowering related genes or coding sequences include TERMINAL FLOWER 1 (tfl1) in Arabidopsis and its homolog CENTRORADIALS (cen) in Antirrhinum; FLOWERING LOCUS C (tic) and the emf gene in Arabidopsis. It is noted that any flower-promotion or flower-related coding sequence(s), the down-regulation of which results in no or reduced sexual reproduction (or total vegetative growth), can be used in the present invention.

Down-regulation of expression of one or more flower promotion or coding sequences in a plant, such as a flo/lfy homolog, results in reduced or no sexual reproduction or total vegetative growth in the transgenic plant, whereby the transgenic plant is unable to produce flowers (or there is a significant delay in flower production). The high conservation observed among flo/lfy homologs indicates that further flo/lfy homologs can be isolated from other plant species by using, for example, the methods of Kyozuka et al. (Proc. Natl. Acad. Sci. 95:1979-1982 (1998)) and Bomblies et al. (Development 130:2385-2395 (2003)). For example, the flo/lfy homolog from bentgrass (Agrostis stolonifera L.) has been cloned (U.S. Patent Application No. 2005/0235379).

Accordingly, in some embodiments of the present invention, RNAi technology can be used to turn off the expression of one or more endogenous genes involved in the transition from a vegetative to a reproductive growth stage, as set forth above.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides.

Nucleic acids of this invention can comprise a nucleotide sequence that can be identical in sequence to the sequence which is naturally occurring or, due to the well-characterized degeneracy of the nucleic acid code, can include alternative codons that encode the same amino acid as that which is found in the naturally occurring sequence. Furthermore, nucleic acids of this invention can comprise nucleotide sequences that can include codons which represent conservative substitutions of amino acids as are well known in the art, such that the biological activity of the resulting polypeptide and/or fragment is retained. A nucleic acid of this invention can be single or double stranded. Additionally, the nucleic acids of this invention can also include a nucleic acid strand that is partially complementary to a part of the nucleic acid sequence or completely complementary across the full length of the nucleic acid sequence. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA or antisense RNA. Genes may or may not be capable of being used to produce a functional protein. Genes include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

An “isolated” nucleic acid of the present invention is generally free of nucleic acid sequences that flank the nucleic acid of interest in the genomic DNA of the organism from which the nucleic acid was derived (such as coding sequences present at the 5′ or 3′ ends). However, the nucleic acid of this invention can include some additional bases or moieties that do not deleteriously affect the basic structural and/or functional characteristics of the nucleic acid. “Isolated” does not mean that the preparation is technically pure (homogeneous).

The term “transgene” as used herein, refers to any nucleic acid sequence used in the transformation of a plant or other organism. Thus, a transgene can be a coding sequence, a non-coding sequence, a cDNA, a gene or fragment or portion thereof, a genomic sequence, a regulatory element and the like.

The term “antisense” or “antigene” as used herein, refers to any composition containing a nucleotide sequence that is either fully or partially complementary to, and hybridize with, a specific DNA or RNA sequence. The term “antisense strand” is used in reference to a nucleic acid strand that is complementary to the “sense” strand. Antisense molecules include peptide nucleic acids (PNAs) and may be produced by any method including synthesis, restriction enzyme digestion and/or transcription. Once introduced into a cell, the complementary nucleic acid sequence combines with nucleic acid sequence(s) present in the cell (e.g., as an endogenous or exogenous sequence(s)) to form a duplex thereby preventing or minimizing transcription and/or translation. The designation “negative” is sometimes used in reference to the antisense strand, and “positive” is sometimes used in reference to the sense strand. An antigene sequence can be used to form a hybridization complex at the site of a noncoding region of a gene, thereby modulating expression of the gene (e.g., by enhancing or repressing transcription of the gene).

The term “RNAi” refers to RNA interference. The process involves the introduction of RNA into a cell that inhibits the expression of a gene. Also known as RNA silencing, inhibitory RNA, and RNA inactivation. RNAi as used herein includes double stranded (dsRNA), small interfering RNA (siRNA), small hairpin RNA (or short hairpin RNA) (shRNA) and microRNA (miRNA).

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

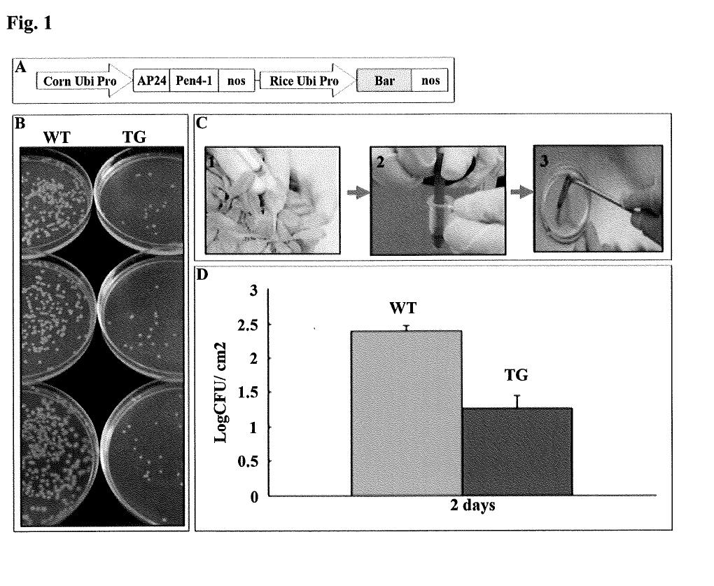

Various nonlimiting examples of a nucleic acid construct of this invention are provided in FIGS. 1-7. Particular embodiments of this invention comprise, consist essentially of and/or consist of the following nucleic acid constructs.

A nucleic acid construct of this invention can comprising in the following order from 5′ to 3′: a) a first promoter; b) a signal peptide sequence; c) a nucleotide sequence encoding an AMP derived from an animal; d) a linker sequence; e) a nucleotide sequence encoding and AMP derived from a plant; f) a first termination sequence; g) a second promoter sequence; h) a selectable marker coding sequence; and i) a second termination sequence.

A nucleic acid construct of this invention can comprise, in the following order from 5′ to 3′: a) a first promoter; b) a signal peptide sequence; c) a nucleotide sequence encoding an AMP derived from a plant; d) a linker sequence; e) a nucleotide sequence encoding an AMP derived from an animal; f) a first termination sequence; g) a second promoter sequence; h) a selectable marker coding sequence; and i) a second termination sequence.

A nucleic acid construct of this invention can comprise in the following order from 5′ to 3′: a) a corn ubiquitin promoter; b) an AP24 signal peptide sequence; c) a nucleotide sequence encoding PEN4-1; d) an IbAMP propeptide coding sequence; e) a nucleotide sequence encoding IbAMP-4; f) a first nos sequence; g) a rice ubiquitin promoter sequence; h) a bar coding sequence; and i) a second nos sequence.

A nucleic acid construct of this invention can comprise, in the following order from 5′ to 3′: a) a corn ubiquitin promoter; b) an AP24 signal peptide sequence; c) a nucleotide sequence encoding IbAMP4; d) an IbAMP propeptide coding sequence; e) a nucleotide sequence encoding PEN4-1; f) a first nos sequence; g) a rice ubiquitin promoter sequence; h) a bar coding sequence; and i) a second nos sequence.

A nucleic acid construct of this invention can comprise, in the following order from 5′ to 3′: a) a corn ubiquitin promoter; b) an AP24 signal peptide sequence; c) a nucleotide sequence encoding PEN4-1; d) a first nos sequence; e) a rice ubiquitin promoter sequence; f) a bar coding sequence; and g) a second nos sequence.

A nucleic acid construct of this invention can comprise, in the following order from 5′ to 3′: a) a corn ubiquitin promoter; b) an AP24 signal peptide sequence; c) a nucleotide sequence encoding IbAMP4; d) a first nos sequence; e) a rice ubiquitin promoter sequence; f) a bar coding sequence; and g) a second nos sequence.

The present invention also provides a nucleic acid construct of this invention, comprising in the following order from 5′ to 3′: a) a first promoter sequence; b) an antisense sequence directed to an endogenous gene involved in plant reproduction; c) a linker sequence; d) a sense sequence complementary to the antisense sequence of (b); e) a first termination sequence; f) a second promoter sequence; g) a signal peptide sequence; h) a nucleotide sequence encoding an AMP derived from an animal, an AMP derived from a plant or both; i) a second termination sequence; j) a third promoter sequence; k) a selectable marker coding sequence; and 1) a third termination sequence.

The present invention also provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding PEN4-1; i) a second nos sequence; j) a rice ubiquitin promoter sequence; k) a bar coding sequence; and 1) a third nos sequence.

In addition, the present invention provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding Ib-AMP-4; i) a second nos sequence; j) a rice ubiquitin promoter sequence; k) a bar coding sequence; and 1) a third nos sequence.

The present invention also provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding PEN4-1; i) an IbAMP propeptide coding sequence; j) a nucleotide sequence encoding IbAMP4; k) a second nos sequence; 1) a rice ubiquitin promoter sequence; m) a bar coding sequence; and n) a third nos sequence.

The present invention additionally provides a nucleic acid construct of this invention, comprising in the following order from 5′ to 3′: a) a CaMV 35S promoter sequence; b) a FLO/LFY antisense sequence; c) a GUS linker sequence; d) a FLO/LFY sense sequence; e) a first nos sequence; f) a corn ubiquitin promoter; g) an AP24 signal peptide sequence; h) a nucleotide sequence encoding IbAMP4; i) an IbAMP propeptide coding sequence; j) a nucleotide sequence encoding PEN4-1; k) a second nos sequence; 1) a rice ubiquitin promoter sequence; m) a bar coding sequence; and n) a third nos sequence.

The elements of the nucleic acid constructs of the present invention can be in any combination. Thus, in the nucleic acid constructs described above, with the elements defined alphabetically as being in the order listed, the respective elements can be present in the order described and immediately adjacent to the next element upstream and/or downstream, with no intervening elements and/or the respective elements can be present in the order described and intervening elements can be present between the elements, in any combination.

In addition, in the constructs of this invention that recite multiple elements of the same name (e.g., a first promoter and a second promoter or a first termination sequence and a second termination sequence or a first nucleotide sequence encoding an AMP and a second nucleotide sequence encoding an AMP) in a single construct, such similarly named elements can be the same or they can be different in any combination (e.g., a first promoter sequence can be a corn ubiquitin promoter sequence and a second promoter sequence can be rice ubiquitin promoter sequence or a first termination sequence can be nos and a second termination sequence can also be nos).

The present invention further provides a transformed plant cell comprising the nucleic acid construct or a multiplicity of different nucleic acid constructs of this invention, in any combination. Furthermore, the elements of the nucleic acid constructs transformed into the plant cell can be in any combination.

A transgenic plant is also provided herein, comprising, consisting essentially of and/or consisting of one or more nucleic acid constructs of this invention. A transgenic plant is additionally provided herein comprising a transformed plant cell of this invention.

Additionally provided herein is a transgenic seed, a transgenic pollen grain and a transgenic ovule of the transgenic plant of this invention. Further provided is a tissue culture of regenerable transgenic cells of the transgenic plant of this invention.

A plant of this invention can be an angiosperm, a gymnosperm, a bryophyte, a fern and a fern ally. In some embodiments the plant is a dicot and in some embodiments, the plant is a monocot. In some embodiments, the plant of this invention is a crop plant.

Nonlimiting examples of a plant of this invention include, turfgrass (e.g., creeping bentgrass, tall fescue, ryegrass), forage grasses (e.g., Medicago trunculata, alfalfa), switchgrass, trees (e.g., orange, lemon, peach, apple, plum, poplar, coffee), tobacco, tomato, potato, sugar beet, pea, green bean, lima bean, carrot, celery, cauliflower, broccoli, cabbage, soybean, oil seed crops (e.g., canola, sunflower, rapeseed), cotton, Arabidopsis, pepper, peanut, grape, orchid, rose, dahlia, carnation, cranberry, blueberry, strawberry, lettuce, cassaya, spinach, lettuce, cucumber, zucchini, wheat, maize, rye, rice, flax, oat, barley, sorghum, millet, sugarcane, peanut, beet, potato, sweetpotato, banana, and the like.

Nonlimiting examples of the types of pathogens against which a transgenic plant of this invention can have increased or enhanced resistance include plant pathogenic fungi, plant pathogenic bacteria, plant pathogenic viruses, plant pathogenic nematodes, plant pathogenic spiroplasmas and mycoplasma-like organisms and plant pathogenic water molds. Nonlimiting examples of a fungal pathogen against which a transgenic plant of this invention can have increased or enhanced resistance include Alternaria spp. (e.g. A. longipes, A alternata, A. solani, A. dianthi), Botrytis spp. (e.g., B. cinerea, B. tulipae, B. aclada, B. anthophila, B. elliptica), Cercospora spp. (e.g., C. asparagi, C. brassicicola C. apii), Claviceps spp. (C. purpurea, C. fusiformis), Cladosporium spp. (e.g., C. sphaerospermum, C. fuivum, C. cucumerinum), Fusarium spp. (e.g., F. oxysporum, F. moniliforme, F. solani, F. culmorum, F. graminearum), Helminthosporium, spp. (e.g., H. solani, H. oryzae, H. Victoriae), Cochliobolus spp., Dreschlera spp., Penicillium spp. (e.g., P. digitatuin, P. expansum), Trichoderma spp. (T. viride, T. hamatum), Verticillium spp. (e.g., V. alboatrum, V. dahliae, V. fungicola), Colletotrichum spp. (e.g., C. gloeosporioides, C. lagenarium, C. coccodes, C. orbiculare), Gloeodes spp. (e.g., G. Pomigena), Glomerella spp. (e.g., G. cingulata, G. glycines), Gloeosporium solani, Marssonina spp. (e.g., M. populi), Nectria spp. (e.g, N. galligena, N. cinnabarina), Phialophora malorum, Sclerotinia spp. (e.g., S. sclerotiorum, S. trifoliorum), Magneporthe spp. (e.g., M. grisea, M. salvinii), Rhizoctonia spp. (R. Solani), Mycosphaerella spp. (e.g., M. fijiensis, M. dianthi. M. citri, M. graminicola), Ustilago spp. (e.g., U. maydis)

Nonlimiting examples of a bacterial pathogen against which a transgenic plant of this invention can have increased or enhanced resistance include Pseudomonas spp. (e.g., P. syringae, P. syringae pv. Tabaci, P. marginata), Erwinia spp. (E. carotovora, E. amylovora), Xanthomonas spp., and Agrobacterium spp. (A. tumefaciens, A. rhizogenes), and the like.

Nonlimiting examples of a water mold which a transgenic plant of this invention can have increased or enhanced resistance include Pythium spp. (P. aphanidermatum, P. graminicola, P. ultimatum), Phytophthora spp. (e.g., P. citrophthora, P. infestans, P. cinnamomi, P. megasperma, P. syringae).

Nonlimiting examples of a nematode which a transgenic plant of this invention can have increased or enhanced resistance include Xiphenema spp. (X. americanum), Pratylenchus spp. (P. neglectus, P. thornei), Paratylenchus spp. (P. bukowinensis), Criconemella spp. (C. xenoplax, C. curvata; C. ornata), Meloidogyne spp. (M. incognita, M. graminicola, M. arenaria), Helicotylenchus spp. (H. dihystera, H. multicinctus), Rotylenchulus spp., Longidorus spp., Heterodera spp. (H. glycines, H. zeae, H. schachtii), Anguina spp. (A. agrostis, A. tritici), Tylenchulus spp. (T. semipenetrans)

Nonlimiting examples of a virus which a transgenic plant of this invention can have increased or enhanced resistance include Rhabdovirus, Alfamovirus, Tobomovirus, Luteovirus, Potyvirus, Cucumovirus, Nepovirus, Comoviridae, Sobemovirus, Carlavirus, Ilarvirus, Potexvirus, Caulimovirus, and Geminivirus. Further nonlimiting examples of a virus which a transgenic plant of this invention can have increased or enhanced resistance include tomato spotted wilt virus, tobacco rattle virus, tobacco necrosis virus, tobacco ring spot virus, tomato ring spot virus, cucumber mosaic virus, peanut stump virus, alfalfa mosaic virus, maize streak virus, figwort mosaic virus, tomato golden mosaic virus, tomato mottle virus, tobacco mosaic virus, cauliflower mosaic virus, tomato yellow leaf curl virus, tomato leaf curl virus, potato yellow mosaic virus, African cassaya mosaic virus, Indian cassaya mosaic virus, bean golden mosaic virus, bean dwarf mosaic virus, squash leaf curl virus, cotton leaf curl virus, beet curly top virus, Texas pepper virus, Pepper Huastico virus, alfalfa mosaic virus, bean leaf roll virus, bean yellow mosaic virus, cucumber mosaic virus, pea streak virus, tobacco streak virus, and white clover mosaic virus.

Nonlimiting examples of a spiroplasma or mycoplasma-like organism which a transgenic plant of this invention can have increased or enhanced resistance include Phytoplasma spp. (P. oryzae, P. solani, P. trifolii, P. ulmi) and Spiroplasma spp.

Additional embodiments of this invention include methods of producing a transgenic plant and the plants produced according to the methods described herein.

Thus, in one embodiment, the present invention provides a method of producing a transgenic plant having increased resistance to infection by a pathogen, comprising: a) transforming a cell of a plant with one or more (e.g., 2, 3, 4, 5, 6, etc.) nucleic acid constructs of this invention and b) regenerating the transgenic plant from the transformed plant cell, wherein the plant has increased resistance to infection by a pathogen as compared with a plant that is not transformed with said nucleic acid construct(s).

Additionally provided herein is a method of producing a transgenic plant having increased resistance to infection by a pathogen, comprising: a) transforming a cell of a plant with a nucleic acid construct comprising a nucleotide sequence encoding PEN4-1; and b) regenerating the transgenic plant from the transformed plant cell, wherein the plant has increased resistance to bacterial and or fungal infection as compared with a plant that is not transformed with said nucleic acid construct.

Further provided herein is a method of producing a transgenic plant having increased resistance to bacterial and/or fungal infection, comprising: a) transforming a cell of a plant with a nucleic acid construct comprising a nucleotide sequence encoding Ib-AMP4 and a nucleotide sequence encoding PEN4-1; and b) regenerating the transgenic plant from the transformed plant cell, wherein the plant has increased resistance to infection by a pathogen. as compared with a plant that is not transformed with said nucleic acid construct.

Further provided herein is a method of producing a transgenic plant having increased resistance to bacterial and/or fungal infection, comprising: a) transforming a cell of a plant with a first nucleic acid construct comprising a nucleotide sequence encoding Ib-AMP4 and second nucleic acid construct comprising a nucleotide sequence encoding PEN4-1; and b) regenerating the transgenic plant from the transformed plant cell, wherein the plant has increased resistance to infection by a pathogen as compared with a plant that is not transformed with said nucleic acid constructs.

Additional embodiments of this invention comprise a method of producing an AMP in a plant, transforming a cell of the plant with one or more nucleic acid constructs of this invention encoding one or more AMPs; b) regenerating the transgenic plant from the transformed plant cell; c) collecting the AMP(s) from the plant. Nonlimiting examples of AMPs that could be produced and collected from the plant using the methods of the present invention are Ib-AMPs, alfAFPs, Pn-AMPs, Mj-AMPs, MiAMPs, pea defensins, penaeidins (e.g., penaeidin 4), apidaecin, magainins, cercropins, lactoferricin tachyplesin, esculentin, polyoxin, nisin, and the like.

Use of plants as platforms for producing commercially valuable heterologous proteins is well-known in the art. See, for example, U.S. Pat. No. 6,040,498; U.S. Patent Application Publication No. 2009/0220543; WO2000/77174; U.S. Pat. No. 7,491,509 and Plants as Factories for Protein Production, eds. E. E. Hood and J. A. Howard, Kluwer Academic Publishers Norwell, Mass., pp 209 (2002). Molecular farming: plant-made pharmaceuticals and technical proteins, eds. R. Fischer and S. Schillberg; Wiley-VCH Verlag GmbH & Co. CGaA, Wienheim (2004).

The process of producing heterologous proteins from plants requires an initial choice of a plant system in which to express the heterologous protein(s) of interest. Many plants have been shown to be amenable to transformation via a wide variety of techniques. Non-limiting examples of transformable plants include tobacco, corn, Arabidopsis, soybean, cotton, carrot, asparagus, rice, turfgrass, lettuce, spinach, white clover, alfalfa, peanut, sunflower, canola, duckweed, wheat, cassava, sugar cane and the like. Expression of heterologous proteins in plants can be accomplished either by integrating the gene of interest into a plant genome, to create a transgenic plant that stably expresses the desired protein, or by introducing the gene of interest into a plant vector that can be introduced into, and transiently maintained in, plant cells. Once the plant is transformed and the production of the heterologous protein(s) is at a sufficient level, the plants can be harvested and the protein(s) collected and purified. Methods for collection and purification of proteins from plants are known in the art (See, e.g., WO2000/77174; U.S. Pat. No. 5,981,835; U.S. Pat. No. 6,846,968 and U.S. Application Publication No. 2005/0015830)

The term “transformation” as used herein refers to the introduction of a heterologous nucleic acid into a cell. Transformation of a cell may be stable or transient. The term “transient transformation” or “transiently transformed” refers to the introduction of one or more heterologous nucleic acids into a cell wherein the heterologous nucleic acid is not heritable from one generation to another.

“Stable transformation” or “stably transformed” refers to the integration of the heterologous nucleic acid into the genome of the plant or incorporation of the heterologous nucleic acid into the cell or cells of the plant (e.g., via a plasmid) such that the heterologous nucleic acid is heritable across repeated generations. Thus, in one embodiment of the present invention a stably transformed plant is produced.

Transient transformation may be detected by, for example, an enzyme-linked immunosorbent assay (ELISA) or Western blot, which can detect the presence of a peptide or polypeptide encoded by one or more transgene introduced into a plant. Stable transformation of a cell can be detected by, for example, a Southern blot hybridization assay of genomic DNA of the cell with nucleic acid sequences which specifically hybridize with a nucleotide sequence of a transgene introduced into a plant. Stable transformation of a cell can be detected by, for example, a Northern blot hybridization assay of RNA of the cell with nucleic acid sequences which specifically hybridize with a nucleotide sequence of a transgene introduced into a plant. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A nucleotide sequence of this invention can be introduced into a plant cell by any method known to those of skill in the art. Procedures for transforming a wide variety of plant species are well known and routine in the art and described throughout the literature. Such methods include, but are not limited to, transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, electroporation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Bacterial mediated nucleic acid delivery includes but is not limited to DNA delivery by Agrobacterium spp. and is described, for example, in Horsch et al. (Science 227:1229 (1985); Ishida et al. (Nature Biotechnol. 14:745 750 (1996); and Fraley et al. (Proc. Natl. Acad. Sci. 80: 4803 (1983)). Transformation by various other bacterial species is described, for example, in Broothaerts et al. (Nature 433:629-633 (2005)).

Physical delivery of nucleotide sequences via microparticle bombardment is also well known and is described, for example, in Sanford et al. (Methods in Enzymology 217:483-509 (1993)) and McCabe et al. (Plant Cell Tiss. Org. Cult. 33:227-236 (1993)).

Another method for physical delivery of nucleic acid to plants is sonication of target cells. This method is described, for example, in Zhang et al. (Bio/Technology 9:996 (1991)). Nanoparticle-mediated transformation is another method for delivery of nucleic acids into plant cells (Radu et al., J. Am. Chem. Soc. 126: 13216-13217 (2004); Torney, et al. Society for In Vitro Biology, Minneapolis, Minn. (2006)). Alternatively, liposome or spheroplast fusion can be used to introduce nucleotide sequences into plants. Examples of the use of liposome or spheroplast fusion are provided, for example, in Deshayes et al. (EMBO J., 4:2731 (1985), and Christou et al. (Proc Natl. Acad. Sci. U.S.A. 84:3962 (1987)). Direct uptake of nucleic acid into protoplasts using CaCl₂ precipitation, polyvinyl alcohol or poly-L-ornithine is described, for example, in Hain et al. (Mol. Gen. Genet. 199:161 (1985)) and Draper et al. (Plant Cell Physiol. 23:451 (1982)). Electroporation of protoplasts and whole cells and tissues is described, for example, in Donn et al. (In Abstracts of VIIth International Congress on Plant Cell and Tissue Culture IAPTC, A2-38, p 53 (1990); D'Halluin et al. (Plant Cell 4:1495-1505 (1992)); Spencer et al. (Plant Mol. Biol. 24:51-61 (1994)) and Fromm et al. (Proc. Natl. Acad. Sci. 82: 5824 (1985)). Polyethylene glycol (PEG) precipitation is described, for example, in Paszkowski et al. (EMBO J. 3:2717 2722 (1984)). Microinjection of plant cell protoplasts or embryogenic callus is described, for example, in Crossway (Mol. Gen. Genetics 202:179-185 (1985)). Silicon carbide whisker methodology is described, for example, in Dunwell et al. (Methods Mol. Biol. 111:375-382 (1999)); Frame et al. (Plant J. 6:941-948 (1994)); and Kaeppler et al. (Plant Cell Rep. 9:415-418 (1990)).

In addition to these various methods of introducing nucleotide sequences into plant cells, expression vectors and in vitro culture methods for plant cell or tissue transformation and regeneration of plants are also well known in the art and are available for carrying out the methods of this invention. See, for example, Gruber et al. (“Vectors for Plant Transformation” in Methods in Plant Molecular Biology and Biotechnology, Glick, B. R. and Thompson, J. E., Eds. (CRC Press, Inc., Boca Raton, (1993), pages 89-119).

The term “vector” refers to a composition for transferring, delivering or introducing a nucleic acid (or nucleic acids) into a cell. A vector comprises a nucleic acid comprising the nucleotide sequence to be transferred, delivered or introduced. In some embodiments, a vector of this invention can be a viral vector, which can comprise, e.g., a viral capsid and/or other materials for facilitating entry of the nucleic acid into a cell and/or replication of the nucleic acid of the vector in the cell (e.g., reverse transcriptase or other enzymes which are packaged within the capsid, or as part of the capsid). The viral vector can be an infectious virus particle that delivers nucleic acid into a cell following infection of the cell by the virus particle.

A plant cell of this invention can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

A large variety of plants have been shown to be capable of regeneration from transformed individual cells to obtain transgenic plants. Those of skill in the art can optimize the particular conditions for transformation, selection and regeneration according to these art-known methods. Factors that affect the efficiency of transformation include the species of plant, the tissue infected, composition of the medium for tissue culture, selectable marker coding sequences, the length of any of the steps of the methods described herein, the kinds of vectors, and/or light/dark conditions. Therefore, these and other factors can be varied to determine the optimal transformation protocol for any particular plant species. It is recognized that not every species will react in the same manner to the transformation conditions and may require a slightly different modification of the protocols disclosed herein. However, by altering each of the variables according to methods routine in the art, an optimum protocol can be derived for any plant species.

Accordingly, in one embodiment, a heterologous nucleotide sequence is introduced into a cell of a plant of the present invention by co-cultivation of the cell with Agrobacterium tumefaciens to produce a transgenic plant. In a further embodiment, a heterologous nucleotide sequence is introduced into a cell of a plant of the present invention by direct nucleic acid transfer to produce a transgenic plant.

### EXAMPLES

### Example 1

### Project Summary for Pen4-1 and IbAMP4 Studies

Turfgrass are highly susceptible to a wide range of destructive fungal and bacteria pathogens, resulting in a great decrease in quality and safety. Chemical pesticides not only add significant operational costs but also raise serious environmental problems. Therefore, the development of disease-resistant turfgrass will not only improve turfgrass quality and reduce turfgrass management costs, but it will also significantly benefit the environment. The major objectives of these studies are to genetically engineer disease resistance in turfgrass through overexpression of the Pen4-1 gene from shrimp (Litopenaeus setiferus) and the Ib-AMP4 gene from balsamine (Impatiens balsamina), respectively, or simultaneously and in some embodiments, in combination with inducing total sterility in transgenic plants through down-regulating the turfgrass FLO/LFY homolog, using RNA interference technology to eliminate gene flow through pollen grains and seeds.

### Experimental Approach

1. Design and synthesize two pSB 11-based Agrobacterium binary vectors for creeping bentgrass transformation with the chimeric gene constructions consisting of an antimicrobial peptide gene (Pen4-1 or Ib-AMP4) or the combination of the two antimicrobial peptide genes under the control of maize uniquitin promoter, a bar gene under the control of rice ubiquitin promoter and a RNAi construction using the creeping bentgrass FLO/LFY homolog that regulates the vegetative to reproductive developmental transition of meristems under the control of a CaMV 35S promoter.

2. Produce transgenic creeping bentgrass lines with the constructs described above via Agrobacterium-mediated transformation.

3. Evaluate the disease resistance of the transgenic plants expressing these two novel antimicrobial genes Pen4-1 and Ib-AMP4.

4. Evaluate efficacy of the total sterility strategy in the prevention of transgene escape from genetically modified grass through “pollen cage” studies.

5. Examine the roles of the introduced antimicrobial peptide genes in the proposed signaling defense pathways by reverse transcriptase-polymerase chain reaction (RT-PCR) and Northern analysis and the resulting global gene expression change by microarray analysis.

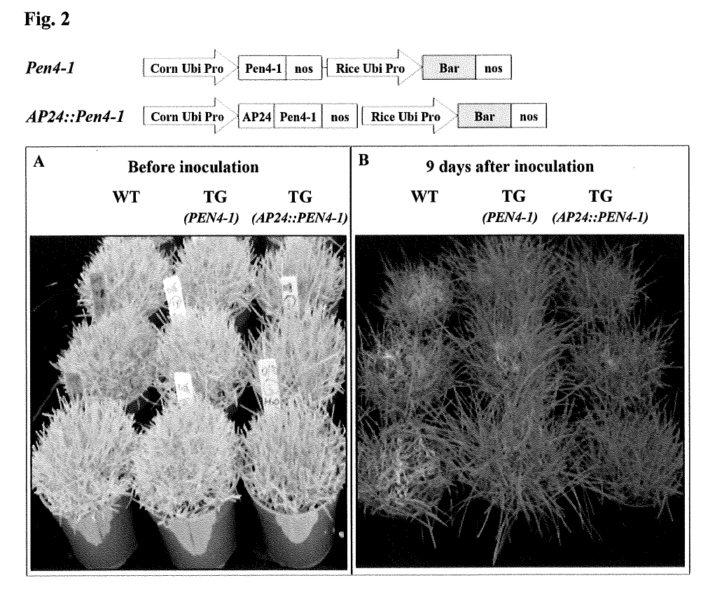

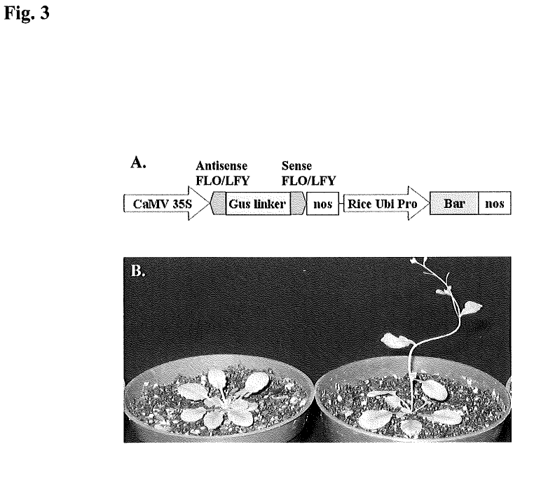

Application of Ib-AMP4 and Pen4-1 in plants. The feasibility of using the Pen4-1 and Ib-AMP4 genes in turfgrass for improved plant response to disease using a transgenic approach has been evaluated. These studies have demonstrated that transgenic plants of Arabidopsis and creeping bentgrass overexpressing the Pen4-1 gene displayed dramatically enhanced disease resistance (FIGS. 1 and 2).

Potential environmental risks of genetically modified (GM) turfgrass. When employing genetic engineering in plants for trait modifications, the possibility of transgene escape to wild and non-transformed species raises various ecological concerns regarding commercialization of transgenic turfgrass. Although numerous risk assessment studies have been conducted on transgenic plants of annual and/or self-pollinating crops, little information is available on the potential risks from large-scale production of outcrossing transgenic turfgrasses (Ainley, 2000; Snow et al, 2002).

In one study, Reichman et al. (2006) monitored a pollen or seed transfer study from a large field of genetically modified (GM) herbicide-tolerant creeping bentgrass (Agrostis stolonifera L.) to look at cp4 epsps transgene flow and escape. The results from this study demonstrated that transgene flow from short-term production to closely related grass species can result in establishment of transgenic plants at multi-kilometer (up to 3.8 km) distances from GM source fields (Reichman et al., 2006). This experiment provided the first evidence of transgene escape to wild populations in perennial species in the US. Later, Zapiola et al. (2008) monitored a 4-year study on escape and establishment of transgenic glyphosate-resistant creeping bentgrass (GRCB) in Oregon. It was found that although all the practices of GRCB fields were strictly regulated, evidence of transgene flow was found all year. Moreover, even 3 years after taking out the production of GRCB, 62% out of 585 creeping bentgrass plants tested in situ were glyphosate-resistant. These authors confirmed that “it was unrealistic to think that containment or eradication of GRCB could be accomplished.” (Zapiola et al., 2008).

The data suggest that in order to release GM grass into agronomic habitats including golf courses and parks, concerns must be addressed about transgene flow from GM grass to other compatible grass or weed species under natural ecological conditions. Therefore, a proper transgene containment strategy should be introduced into large scale production of transgenic turfgrass species.

Introduction to general plant transgene containment strategies: In flowering plants, gene flow can occur through movement of pollen grains and seeds. Various gene containment strategies have been developed to alter gene flow by interfering with flower pollination, fertilization, or fruit development. One approach, which is largely used in crop plants such as rice and corn, is to interfere with the development of male reproductive structures through genetic engineering to cause male sterility. For example, tapetum-specific expression of cytotoxic molecules blocks pollen development, which has been recently used in transgenic creeping bentgrass for preventing transgene flow through pollen (Luo et al., 2005). However, the efficacy of male sterility in the prevention of transgenic flow under the open-pollinated field conditions remains to be determined (Luo et al., unpublished), and the seeds produced from male-sterile GM crops by cross-pollination from weeds may pose serious problems.

Maternal inheritance is another approach for trans gene containment, with added advantages of high levels of transgene expression, rapid multigene engineering, lack of position effects and gene silencing (Daniell, 2004). Currently, chloroplast genetic engineering has been shown to be efficacious in tobacco to confer desirable plant traits or work as a bioreactor for production of biopharmaceuticals. It has also been used in potato and tomato (Daniell, 2004). However, chloroplast transformation in monocot species is still immature.

Other strategies like seed-sterility, cleistogamy, apomixes and genome incompatibility are still at the exploratory stage (Daniell, 2004) and not ready for perennial turfgrass species. Therefore a suitable strategy for transgene containment that is desirable for perennial turf grass species is needed.

Total sterility resulting from down-regulation of FLO/LFY homologs: An approach of total sterility resulting from down-regulating plant genes that determine reproductive growth to develop total vegetative growth, eliminating gene flow through movement of both pollen grains and seeds, may satisfy this need for developing a gene containment strategy for perennial turfgrass.

In recent years, significant progress has been made towards understanding the molecular basis of floral transition. Flowering plants, during their post-embryonic development, experience a series of distinct growth phases, each characterized by the identity of the lateral primordia produced by the shoot apical meristem (SAM). In Arabidopsis, during the vegetative phase the SAM produces leaf primordia that subtend secondary shoot meristems. Later, during the early reproductive phase, subtended auxiliary inflorescence meristems are produced (Poethig, 1990). During the late reproductive phase, floral primordia that will develop into a flower are produced. The transition from vegetative phase to reproductive phase, i.e., the floral transition, is the most dramatic phase change in plant development. This transition is regulated by a complex genetic network (Poethig, 2003). A number of genetic models have proposed that the activation of the floral meristem identity genes, such as LEAFY (LFY) or APETALA1 (AP1), plays an important role in specifying the floral fate of nascent lateral primordia produced by the SAM (Coen et al., 1990; Weigel et al., 1992; Kyozuka et al, 1998; Poethig, 2003).

Genetic and molecular studies have shown that the genetic network controlling flower development in two dicot species, Antirrhinum and Arabidopsis, is conserved. After the transition from vegetative to reproductive phase, floral meristems are initiated by the action of a set of floral meristem identity genes. Among them, FLORICAULA (FLO) of Antirrhinum and its Arabidopsis counterpart LEAFY (LFY), which encode transcription factor with no significant homology with any known gene, seem to play the most important role in the establishment of floral fate. In strong flo and lfy mutant plants, flowers are transformed into inflorescence shoots (Coen et al., 1990; Weigel et al., 1992).

Gramineae is a large and variable family within the monocots. Many features of flower development and mature architecture of grass flowers are distinct from those of dicots. Little is known about molecular mechanisms controlling floral development in grass species compared with dicot species. However, FLO/LFY homologs have been identified in several species, such as rice (Kyozuka et al., 1998); Lolium temulentum and ryegrass (Lolium perenne) (Gocal et al., 2001); and maize (Bomblies et al., 2003). Zfl (Zfl1 and Zfl2), the FLO/LFY homolog of maize appears to share the function with LFY/FLO of Arabidopsis/Antirrhinum, as the zfl double mutants have been characterized as having defective phase transitions. RFL, the rice FLO/LEF homolog, has been isolated and analyzed. Its expression and function indicate that it partially conserves the FLO/LFY function of Arabidopsis/Antirrhinum. In general, the principle functions of FLO/LFY are largely conserved in flowering plants with regard to phase transition. (Coen et al., 1990; Weigel et al., 1992; Kyozuka et al., 1998; Gocal et al., 2001; Bomblies et al., 2003).

In addition, the DNA sequences and amino acid sequences of LFY/FLO homologs from different species have high homology especially in C-terminal regions. These important characteristics of the FLO/FLY-like genes in plants strongly indicate that if the expression of the corresponding genes in turfgrass is turned off, the genetically modified plants grown in the field will maintain total sterility, thus eliminating any potential risks of transgene escape through the reproductive pathway.

Because the FLO/LFY homologs in different species have high sequence homology at the DNA level, primers can be designed in the well conserved regions. A 250-bp DNA fragment of FLO/LFY homolog from creeping bentgrass has been amplified through PCR. Studies in which this 250-bp fragment is used as a probe to conduct Southern blot analysis of creeping bentgrass genomic DNA have shown that the gene is present as a single copy. Studies in which an RNAi construct in which this 250-bp fragment of sense and anti-sense LFY/FLO homologs is linked by GUS fragment and introduced into the Arabidopsis plants (FIG. 3) and creeping bentgrass plants have been carried out. Creeping bentgrass plants transformed with this RNAi construct failed to transition from vegetative to reproductive growth and thus, remained in vegetative growth without producing flowers.

These important results provided the basis for the current studies to achieve the goals of incorporation of total sterility into transgenic creeping bentgrass plants with improved disease resistance to produce environmentally friendly transgenic plants for commercialization.

Potential effects of these two novel antimicrobial genes on PR gene expression and global gene expression. In response to microbial attacks, plants activate a complex series of responses leading to the local and systemic induction of a wide range of antimicrobial defenses which include strengthening of mechanical barriers, oxidative burst, and production of antimicrobial compounds. If the induced defense responses are rapidly and coordinately triggered in confronting microbial challenges, plants become resistant to diseases (Kim and Martin, 2004; Park, 2005; Lee et al., 2009). In the past few decades, much research has focused on the activation and the production of antimicrobial compounds, and their roles in plant defense pathways contributing to the outcomes of plant-pathogen interactions (Selitrennikoff, 2001; Lee et al., 2009). The present study investigates the roles of the constitutively overexpressed antimicrobial peptides Pen4-1 or Ib-AMP4 on the defense response pathway, in addition to their direct antimicrobial effects in transgenic resistant plants.

A number of signaling molecules such as salicylic acid (SA), ethylene, and jasmonic acid (JA), are known to regulate defense responses in plants during initial activation events. They can trigger the oxidative burst and expression of PR proteins. The accumulation of PR proteins is intimately correlated with plant disease resistance (Lee and Hwang, 2005). The present studies analyze whether the antimicrobial peptides Pen4-1 or Ib-AMP4 have effects on mediating these signaling molecules, thus impacting the activation and production of PR proteins.

In Arabidopsis, the signaling molecule, SA, is intimately associated with biotrophic pathogen infection such as with the bacterial strain, Pst DC3000, and is necessary for systemic required resistance in plants (Mishina and Zeier, 2007; Lee et al., 2009). SA is also known to regulate the expression of different sets of PR genes, such as PR1 and PR5 (Gu et al., 2002). Data in FIG. 1 show that Pen4-1-OX Arabidopsis plants are resistant to the virulent strain of bacteria Pst DC3000. Expression of Pen4-1 may result in a more rapid induction of SA, which impacts the activation and expression of PR genes. To examine the AtPR1 and AtPR5 gene expression changes under normal and microbial challenge conditions, RT-PCR analyses will be used.

The model organism employed in these studies is rice. In rice, SA can induce the PR protein, OsPR10 (Jwa et al., 2001). A probe will be designed for Northern analysis based on the sequence of OsPR10 in rice to hybridize with total RNA from wild-type and transgenic creeping bentgrass plants under normal and microbial challenge conditions to determine whether Pen4-1 and Ib-AMP4 are involved in the SA signaling pathway.

The antimicrobial peptides Pen4-1 and Ib-AMP4 may not only interact with SA but also with other signaling molecules such as JA and ethylene. Moreover, SA itself will also cross talk with other signaling molecules, such as MeJA (Liu et al., 2005). In this case, the activation and expression level of PR proteins working in other signaling pathways, for example, PR1a and PR1b genes which are specifically induced by JA (Agrawal et al., 2000), can be examined.

To more broadly assess the secondary effects of Pen4-1 and Ib-AMP4, microarray analysis will be used to profile the global changes in gene expression in wild-type and transgenic plants in stressed and non-stressed conditions. For Arabidopsis, gene chips will be used directly in a study to analyze the global gene expression profile. For turfgrass, due to the lack of extensive genomics resources, rice gene chips will be used for analysis of the global gene expression profile in wild-type and transgenic turfgrass plants under normal and stressed conditions by heterologous approaches. This approach allows not only for the confirmation of the role of Pen4-1 and Ib-AMP4 in the aforementioned signaling pathways if they are related thereto, but also allows for the identification of their possible roles in other pathways and mechanisms.

Rationale and significance. Turfgrass management and production is one of the fastest growing areas of agriculture (Qu et al., 2008). However, the development of turfgrass disease resistance lags behind and still mainly relies on chemical pesticides, which cause serious problems in plant heath, human health and the environment. Genetic engineering provides the opportunity to incorporate disease resistant traits into turfgrass that are difficult to achieve through traditional breeding. Due to the absence of toxicity to both plants and human health and the high efficiency of antimicrobial capacity, short sequence AMPs-Pen4-1 and Ib-AMP4 have been selected as ideal candidates for genetic engineering. The data herein demonstrates their great performance upon microbial challenges. Moreover, the total sterility transgene containment strategy described herein provides a powerful tool in developing environmentally safe transgenic products. The use of genetic engineering technology to introduce these two novel antimicrobial peptide genes into turfgrass in combination with total sterility strategy will result in an environmentally responsible and economically feasible new turfgrass cultivar with enhanced disease resistance.

To develop new cultivars of turfgrass for enhanced performance upon microbial stress, the genes Pen4-1 and Ib-AMP4 are overexpressed respectively or simultaneously using transgenic techniques to engineer disease resistance into turfgrass. To produce an environmentally friendly turfgrass with great potential for commercialization, down-regulating the turfgrass FLO/LFY homolog using an RNAi approach should prevent expression of the endogenous FLO/LFY homolog, leading to a total vegetative growth without producing any pollen or seeds.

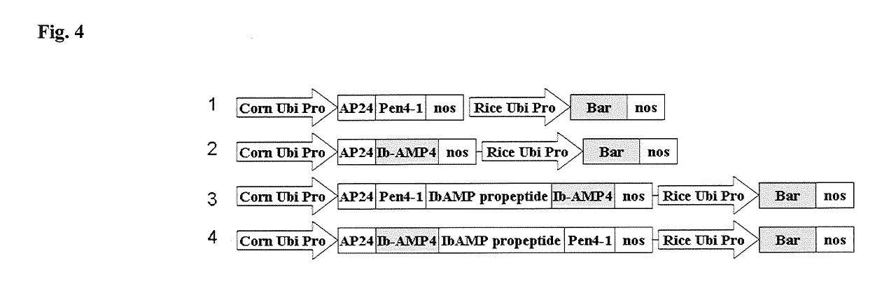

AMP gene constructions. In order to test the efficacy of the chosen AMP genes, Pen4-1 and Ib-AMP4, in fighting against plant diseases, two constructs (FIGS. 4, #1 and #2) have been prepared and introduced into the Agrobacterium tumefaciens strain, LBA4404 (pSB 1), by electroporation. Both constructs include a corn ubiquitin promoter driving an AMP gene fused with the signal peptide of tobacco AP24 antimicrobial gene and a rice ubiquitin promoter driving a bar gene encoding herbicide resistance as a selectable marker, respectively. The function of the AP24 signal peptide is to mediate the transition of the AMP into the endoplasmic reticulum where conditions for disulfide bond formation are favorable and the enzymes that catalyze the reactions are present. Constructs harboring only the AMP gene without the signal peptide sequence have also been prepared and the preliminary data have indicated that the signal peptide will enhance the efficacy of the AMP genes.

Beside the co-transformation strategy to introduce both Pen4-1 and Ib-AMP4 genes into the plants, two polyprotein constructs (FIGS. 4, #3 and #4) have also been prepared, which can produce two different antimicrobial peptides simultaneously. An advantage of using this polyprotein expression strategy is to boost expression levels of small peptides. These prepared polyprotein constructs (FIGS. 4, #3 and #4) include an AP24 signal peptide sequence and two different antimicrobial peptide sequences, Pen4-1 and Ib-AMP4, linked by an intervening sequence (“linker peptide”) originating from a natural polyprotein occurring in the seed of Impatiens balsamina. These chimeric polyproteins are expected to be cleaved in plants and the individual AMPs are to be secreted into the extracellular space. The amount of AMPs produced in plants transformed with these polyprotein transgene constructs is expected to be significantly higher compared to the amount in plants transformed with a single AMP. The AMP genes are positioned in the construct in different order, resulting, e.g., in the different constructs #3 and #4 as shown in FIG. 4.

AMP gene constructions in combination with RNAi constructions. To produce totally sterile creeping bentgrass overexpressing AMP genes, chimeric gene constructs as shown in FIG. 5 will be prepared. As an example of this cloning strategy, in construct #5 as shown in FIG. 5, a fragment of the corn ubiquitin promoter driving the AP24::Pen4-1 fused gene will be released from pSBUbi::AP24::Pen4-1 by HindIII digestion. The Ubi::AP24::Pen4-1 fragment will be blunt-ended by Klenow treatment and ligated into the blunt-ended Avr1 site of pSB35S::RNAi-Ubi::bar, producing the final #5 construct pSB35S::RNAi-Ubi::AP24::Pen4-1-Ubi::bar. The antisense sequence of the construct comprises the creeping bentgrass 3′ end of FLO/LFY homolog as follows: ctacatcaac aagcccaaga tgcggcacta cgtgcactgc tacgcgctgc actgcctgga cgaggaggcc tccgacgcgc tgcgcagggc gtacaaggcc cgcggcgaga acgtcggcgc ctggaggcag gcgtgctacg cgccgctggt ggacatctcc gccaggcacg gcttcgacgt cgacgccgtc ttcgccgcgc acccgcgcct cgccatctgg tacgtgccca ccag (SEQ ID NO:1). The sense sequence comprises the complement of the creeping bentgrass sequence set forth above and is as follows: ctggtgggca cgtaccagat ggcgaggcgc gggtgcgcgg cgaagacggc gtcgacgtcg aagccgtgcc tggcggagat gtccaccagc ggcgcgtagc acgcctgcct ccaggcgccg acgttctcgc cgcgggcctt gtacgccctg cgcagcgcgt cggaggcctc ctcgtccagg cagtgcagcg cgtagcagtg cacgtagtgc cgcatcttgg gcttgttgat gtag (SEQ ID NO:2). The same cloning strategy is applied to the other three constructs of FIG. 5.

The constructed binary vectors prepared as described above will be introduced into Agrobacterium tumefaciens strain, LBA4404 (pSB 1), by electroporation. The resulting Agrobacterium strains will be verified by molecular analysis of plasmid DNA and used for creeping bentgrass transformation via infection of embryogenic callus initiated from mature seeds.

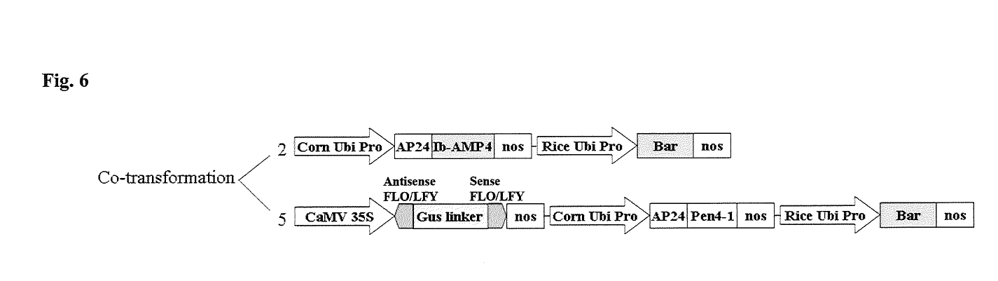

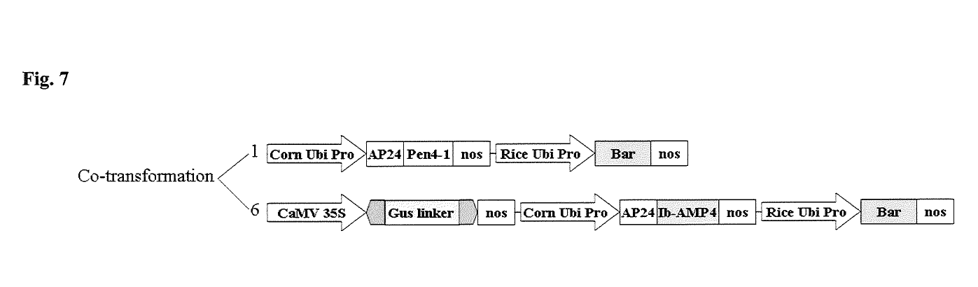

Production of transgenic turfgrass. Creeping bentgrass (Agrostis Stolonifera L. cv. Penn A-4), which is used as turfgrass material in these studies is an important cool weather grass mainly used in golf greens. The aforementioned constructs will be introduced into creeping bentgrass by Agrobacterium-mediated transformation. The same co-transformation strategy is applied to the four constructs #1, #2, #5 and #6 of FIGS. 6-7. In each case, the antisense sequence and sense sequence of the constructs comprise the creeping bentgrass 3′ end of FLO/LFY homolog (antisense) or its complement (sense), as set forth above.

Specifically, constructs #2 and #5 (FIG. 6) will be co-transformed, resulting in transgenic plants harboring a single #2 construct as a control, transgenic plants harboring a single #5 construct and transgenic plants harboring both #2 and #5 constructs. In the same way, constructs #1 and #6 (FIG. 7) will be co-transformed into creeping bentgrass.

The AMP polyprotein plus RNAi structure constructs #7 and #8, will be introduced into plants respectively.

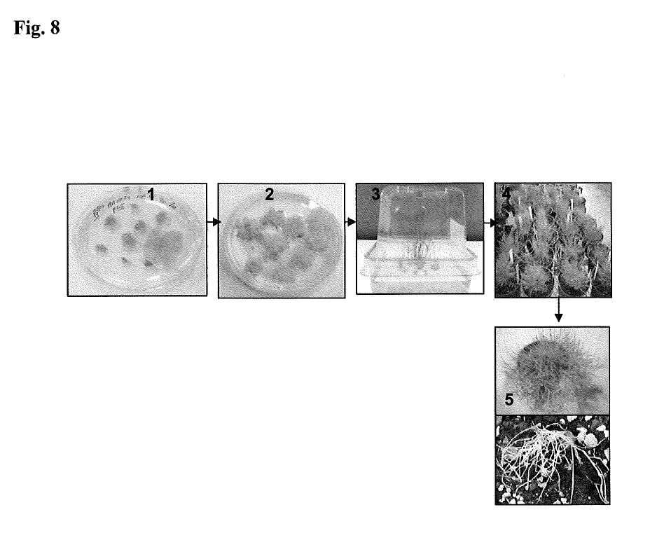

Tissue culture of creeping bentgrass will be carried out in reference to previous procedures (Luo et al, 2004) demonstrated in FIG. 8.

Molecular Analysis. Southern, RT-PCR and Northern analyses will be conducted to confirm the transgene insertion and expression in the transgenic plants.

Bioassay of Transgenic Plants against Different Plant Pathogens. The effects of using these two novel AMP genes for engineering resistance to diseases will be evaluated in the following two series of experiments. The fungal isolates, S. homeocarpa and R. solani, which are destructive turfgrass pathogens, will be used in the experiments.

### 1. In Vivo Test of Direct Plant Inoculation

Preparation of the R. solani and S. homeocarpa cultures and inoculation onto grass will be conducted as described (Wang et al, 2003; Dong et al., 2007). Selected transgenic lines based on molecular analysis will be screened for resistance to S. homeocarpa and R. solani. The wild type creeping bentgrass (Agrostis Stolonifera L. cv. Penn A-4), the non-transformed creeping bentgrass resulting from tissue culture, a transgenic line harboring pSBUbi::RNAi-Ubi::bar (FIG. 3A), and a transgenic line harboring only the AMP construct (FIG. 5), will be included as controls. Pots of each line or the wild type cultivar will be vegetatively replicated by transplanting approximately 20 mature shoots into plastic pots (9×10×10 cm size). Plantlets will be grown in a greenhouse at 25-30° C. for 2 to 4 months. Fertilization, mowing and irrigation will be carried out if needed. Fungal bioassays will be conducted to assess levels of resistance among the transgenic lines against S. homeocarpa and R. solani compared with control plants. The grass will be mowed prior to inoculation, and foliar coverage will be estimated visually as the percentage of area per pot. The center of each pot will then be inoculated with approximately 3 g of colonized inoculum by R. solani and 0.55 g of colonized inoculum by S. homoeocarpa. Plants inoculated with S. homoeocarpa will be placed in plastic containers containing 4 cm of distilled water, and lightly misted with distilled water at 48-h intervals to maintain relative 100% humidity. The containers will be placed inside a greenhouse set to maintain a diurnal cycle of 14 h light and 10 h dark. The creeping bentgrass inoculated with R. solani will be placed in a growth chamber under a 14/10 h (day/night) photoperiod and the temperature and relative humidity will be 30° C. and 70% during daytime, and 24° C. and 95% at night. Ten to sixteen replicates of each transgenic line and wild type cultivar will be used. Each experiment will be repeated three times. Disease severity will be visually estimated at 3, 5 and 7 days post-inoculation using the Horsfall Barrett scale (Horsfall et al., 1945). It is expected that transgenic plants expressing the AMP gene(s) will display enhanced disease resistance.

### 2. Field Test

Field evaluation of the transgenic plants will be carried out according to previously reported procedures (Belanger et al., 2004; Guo et al., 2003). The trial will be established as a randomized complete block design with three vegetatively-propagated replications. The wild type creeping bentgrass, the non-transformed creeping bentgrass resulting from tissue culture, a transgenic line harboring pSBUbi::RNAi-Ubi::bar (FIG. 3A), and a transgenic line harboring only the AMP construct (FIG. 5), will be included as controls. Each replication will consist of 42 plants and maintained as mowed at a height of approximately 2.54 cm with a rotary mower. Weeds will be removed manually as needed. At the point that the 42 plants have grown together to a small area, isolates of S. homoeocarpa or R. solani will be used to inoculate the field. The preparation of the inoculums will be conducted based on previously reported protocols (Guo et al., 2003). Two hundred grams of Kentucky bluegrass (Poa pratensis L.) seeds with 75 ml of dH₂O will be autoclaved for 15 min at 151° C. Fungal isolates will be grown separately on sterilized Kentucky bluegrass seeds. After approximately 3 weeks of growth on the bluegrass seeds, the inoculum will be dried on newspaper for 3 days and forced though a seed sieve. Then the inoculum will be applied to the field with a drop of spreader at a rate of 1.75 g m⁻². Light irrigation will be applied to enhance fungal growth.

Dollar spot symptoms are expected to be observed 2 weeks after inoculation. The weekly rating of the disease severity in each replication area and estimation of the percent of diseased turf will begin. The data obtained from the weekly rating will be subjected to statistical analysis using the Student's t-test. Significance will be evaluated at P<0.05.

Evaluation of the effectiveness of RNAi of the turfgrass FLO/LFY homolog in giving rise to total sterility. Transgenic plants with single copy transgene insertion will be gown in a greenhouse. The wild-type creeping bentgrass, the non-transformed creeping bentgrass resulting from tissue culture, a transgenic line harboring pSBUbi::RNAi-Ubi::bar (FIG. 3A), and a transgenic line harboring only the AMP construct (FIG. 5), will be included as controls. They will be vernalized at 4° C. for 3 months and moved back to the greenhouse for flowering. The morphology of transgenic plants as compared with wild type control will be examined to see how effectively the RNAi construction can knock out endogenous FLO/LFY homolog gene expression in creeping bentgrass and cause total sterility.

According to previous experimental data, this RNAi construct is observed to be able to turn off the LFY gene in Arabidopsis (FIG. 3) and the FLO/LFY homolog in creeping bentgrass. Therefore it is anticipated that the transgenic creeping bentgrass harboring the RNAi constructions will maintain vegetative growth throughout its life cycle.

“Pollen cage” studies on the transgenic plants to evaluate the transgene escape from the GM grass. Since creeping bentgrass is an out-crossing, wind-pollinated, perennial species, two separate experiments will be conducted.

In the first experiment, 20 transgenic plants with total vegetative growth will be arranged to grow together in a cage built with Monofilament Polyester Environmental Microscreening 420 EX-61″. At the same time 20 wild type plants and 20 transgenic plants expressing only AMP gene without RNAi construct will be grown next to each other in a separate cage as positive controls. Upon flowering and maturation, the inflorescences, if any, will be harvested and dried. Seeds from each plant, if any, will be germinated and the number of seedlings will be counted.

In the second experiment, 20 smaller cages will be prepared, and in each of them, one transgenic plant with total vegetative growth will be grown together with a wild type plant. Upon flowering and maturation, the inflorescences, if any, will be harvested and dried. Seeds from each plant, if any, will be germinated and the number of seedlings will be counted. In both experiments, the data obtained will be used for statistical analysis.

Because the transgenic plants will have no reproductive growth, no seed or pollen production will be observed in the transgenic plants in the first experiment. The plants as positive controls are expected to flower normally and produce seeds and pollen. In the second experiment, since the transgenic plants would not produce any flower or pollen and the creeping bentgrass is an out-crossing grass specie, it is expected that no seed production will be observed for either the transgenic plants or the wild type plants. Any self-crossing can be excluded by molecular analysis.

Examination of the possible involvement of PR genes in the enhanced disease resistance of transgenic plants using RT-PCR and Northern analysis. Total RNA will be prepared from Arabidopsis and creeping bentgrass using Trizol RNA extraction buffer (Invitrogen). To analyze gene expression in transgenic Arabidopsis plants by RT-PCR, total RNA (2 μg) from wild-type and transgenic plants will be transcribed using reverse transcriptase (Invitrogen) with oligo(dT) for 1 h at 42° C. PCR will be carried out using two pairs of primers. The primers, 5′-ATGAATTTTACTGGCTTCCAT-3′ (forward) and 5′-AACCCACATGTTCACGGCGGA-3′ (reverse), will be used to amplify the AtPR1 gene. The primers, 5′-TTCACATTCTCTTCCTCGTGTTCA-3′ (forward) and 5′-TCGTAGTTAGCTCCGGTACAAGTG-3′ (reverse), will be used to amplify the AtPR5 gene.

To analyze the level of gene expression in transgenic turfgrass using Northern blotting, the whole process will follow previously published procedures (Lee et al., 2009). The probe will be designed based on the rice OsPR10 gene sequence.

Since the transgenic Arabidopsis plants expressing Pen4-1 gene are resistant to Pst DC3000, which is intimately associated with the SA dependent pathway accompanied with the gene expression change of AtPR1 and AtPR5, the AtPR1 and AtPR5 are expected to be more rapidly and strongly induced by Pst DC3000 infection in transgenic plants than in wild-type plants. Under normal conditions, no significant difference between wild-type and transgenic plants is expected to be observed for the gene expression of AtPR1 and AtPR5, because AtPR1 and AtPR5 expression is more likely to be triggered by indirect effects of the overexpressed transgene, e.g., through SA signals rather than constitutive activation of defense related genes. However, there is the possibility that the enhanced disease resistance is not related to the SA dependent pathway but to some other possible pathway such as a PAMP-triggered resistance response to pathogen attack (Cao et al., 1998; Lee et al., 2009).

Since the transgenic turfgrass plant is resistant to the fungus, S. homeocarpa, the turfgrass homolog OsPR10 is expected to be more rapidly and strongly induced by fungal infection in transgenic plants than in wild-type plants. However, there is also the possibility that the enhanced disease resistance may be related to other response pathways.

Evaluation of global gene expression change under normal and microbial challenge conditions in wild-type and transgenic plants using microarray analysis. To investigate the potential role of Pen4-1 or Ib-AMP4 in contributing to global gene expression in normal and microbial challenge conditions, microarray analysis will be used to profile the global changes in gene expression in wild-type and transgenic plants. For Arabidopsis plants, Arabidopsis GeneChips from Affymetrix will used. Total RNA will be extracted from wild-type and transgenic plants under stressed and non-stressed conditions, respectively. Three biological replicates and three technical replicates will be used. Through transcription and labeling, the processed cRNA will be obtained and used for hybridization to the Arabidopsis Oligonucleotide Array on the Affymetrix Genechip instrument system at Clemson University Genomics Institute Microarray facility. Two sets of data under stressed or non-stressed conditions will be obtained and compared. GeneChip software MAS 5.0 (Affymetrix) will be used for the data analysis.

For creeping bentgrass plants, Rice GeneChips containing 51,279 transcripts from Affymetrix will be used. Total RNA will be extracted from wild-type and transgenic creeping bentgrass plants under stressed and non-stressed conditions, respectively. The finally processed cRNA will be obtained and used for hybridization to the Rice Oligonucleotide Array on the Affymetrix Genechip instrument system at Clemson University Genomics Institute Microarray facility. GeneChip software MAS 5.0 (Affymetrix) and Go program (The Gene Ontology Consortium, 2001) will be used for data analysis.

### Example 2

### Genetic Engineering of Turfgrass with a Novel Antimicrobial Peptide Penaeidin-4 from Litopenaeus setiferus for Enhanced Disease Resistance

Abstract. The antimicrobial peptide Penaeidin4-1 (Pen4-1) from Litopenaeus setiferus has been reported. to possess in vitro antifungal and antibacterial activity against various economically important fungal and bacterial pathogens. We have studied the potential of using this novel peptide for engineering enhanced disease resistance into a commercial turfgrass variety (Agrostis Stolonifera L. cv. Penn A-4). Two DNA constructs were prepared containing coding sequence of a single peptide Pen4-1, and that of a single peptide Pen4-1 fused with a signal peptide of an antimicrobial peptide AP24 from tobacco, respectively. A corn ubiquitin promoter was used in both constructs to drive gene expression. Transgenic turfgrass plants containing different DNA constructs were generated by Agrobacterium-mediated transformation. Transgene insertion and expression was demonstrated. In replicated in vitro and in vivo experiments under controlled environments, resistance was shown to inoculation of isolates of Sclerotinia homoecarpa and Rhizoctoni solani, which can cause the most destructive fungi diseases dollar spot and brown patch to turfgrass. Among those events, transgenic plants transformed with the Pen4-1 gene fused with the AP24 signal peptide sequence exhibited a high performance to brown patch disease. Both transgenic plants transformed with AP24::Pen4-1 gene and single Pen4-1 gene showed a delayed effect to dollar spot disease. In general this novel antimicrobial gene and the strategy of introducing it with AP24 signal peptide sequence may have wide application in various crops.

Introduction. Turfgrass species are highly susceptible to a wide range of fungal pathogens. Dollar spot and brown patch diseases are some of the most severe and frequently occurring diseases on turfgrass lawns in the summer caused by the fungus-Sclerotinia homoecarpa and Rhizoctonia solani, respectively (Chai et al, 2002). Currently, the fungal disease control of turfgrass mainly relies on fungicide treatments. However, the emergence of resistant pathogen strains and the limited spectrum of targets, and the negative long-term impacts on human health and the environment have driven the search for new alternatives for the currently used chemicals. Therefore, in agriculture, there is an urgent requirement to exploit products that present sustainable resistance to a broad range of pathogens and are safe for the host organisms, the consumers and the environment (Zasloff, 2002; Keymanesh, 2009).

Penaeidins, a family of AMPs originally isolated from the haemocytes of penaeid shrimp, are considered to be a source of AMPs which have the potential to be applied in agriculture to deliver disease resistance to plants. Shrimp and other invertebrates lack the adaptive immune system which is characteristic of jawed vertebrates, thus relying exclusively on the innate immune system (Cuthbertson et al, 2006), in which penaeidin antimicrobial peptides are one of the key elements (Cuthbertson et al, 2006). Penaeidins are a diverse peptide family with a unique two-domain structure including an unconstrained proline-rich N-terminal domain (PRD) and a cysteine-rich domain (CRD) with a stable α-helical structure (Cuthbertson et al, 2005). They are primarily directed against Gram-positive bacteria and fungi (Destoumieux et al, 1999) and are synthesized in granular haemocytes, released into the plasma upon microbial infection and localize to tissues, bound to cuticle surfaces (Destoumieux, 2000; Muñoz et al, 2002). The complexity inherent in the multi-domain structure of the peptide may contribute to its broad range of microbial targets (Yang et al, 2003; Destoumieux et al, 2000).

The penaeidin family is divided into four classes, designated 2, 3, 4 and 5 and each class displays a remarkable level of primary sequence diversity (Chen et al, 2004; Cuthbertson et al, 2006). Pen4-1 belongs to class four isoform one of the penaeidins isolated from Atlantic white shrimp (Litopenaeus setiferus). It contains six cysteine residues forming three disulfide bridges, and it is the shortest isoform in penaeidin family with a length of 47 amino acids. It can inhibit multiple plant pathogenic fungal species, including B. cinera, P. crustosum, and F. oxysporum (Bachere et al, 2000). It is also effective against Gram-positive bacteria species including M. luteus and A. viriduans, and it is inhibitory against Gram-negative bacteria such as E. coli at relatively high concentrations (Cuthbertson et al, 2006). Notably, Pen4-1 can fight against multidrug-resistant fungi species: Cryptococcus neoforman (Steroform A, Steroform. B, Steroform C, Steroform D) and Candida spp (Candida lipolytica, Candida inconspicua, Candida krusei, Candida lusitaniae, Candida glabrata (Cuthbertson et al, 2006). Compared with other penaeidins, penaeidin class 4 has shown a high level of effectiveness against fungi (Cuthbertson et al, 2006). Additionally, the unusual amino acid composition of PRD Pen4-1 may confer resistance to proteases (Cuthbertson et al, 2006).

The aim of this study was to investigate the feasibility of using Pen4-1 gene from L. setiferus for engineering resistance to Sclerotinia homoeccarpa into a commercial turfgrass variety (Agrostis Stolonifera L, cv. Penn A-4).

### Synthesis of Pen4-1 Gene

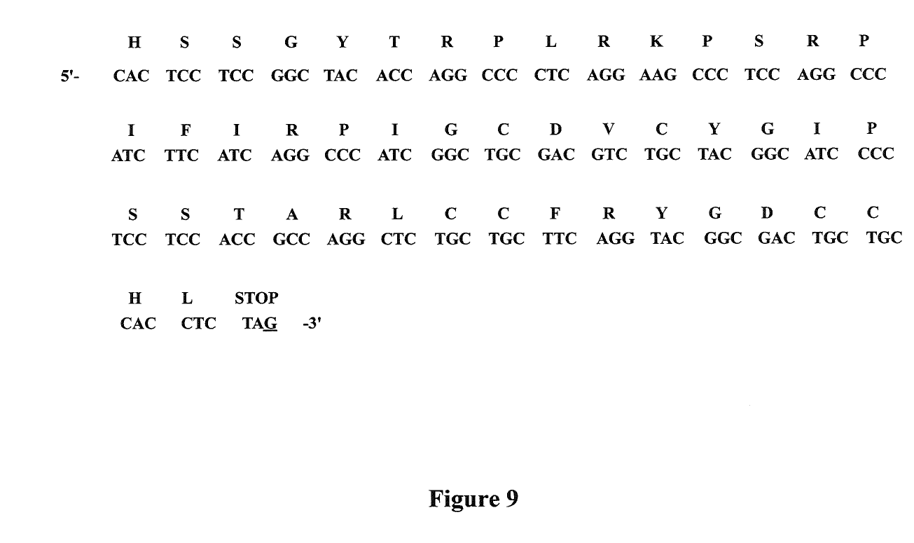

The full sequence of Pen4-1 gene was obtained from Pen-Base. Plant codon preference was done in order to make it friendlier for plant expression. The full sequence of Pen4-1 after codon modification was synthesized in Integrated DNA technology. The amino acid sequences and the modified DNA sequences were demonstrated in FIG. 9.

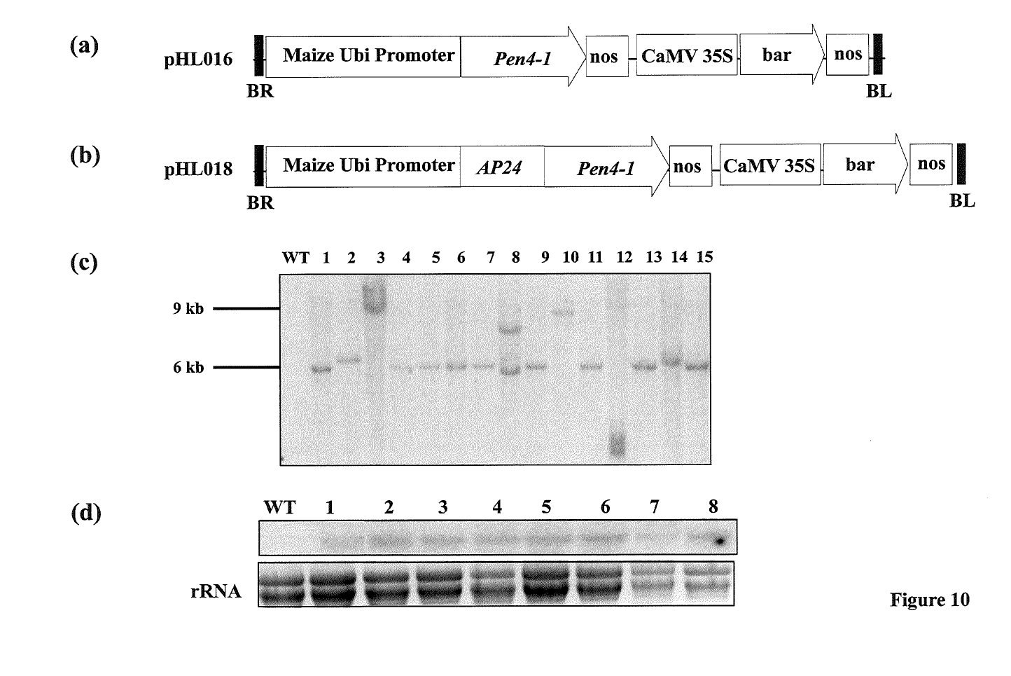

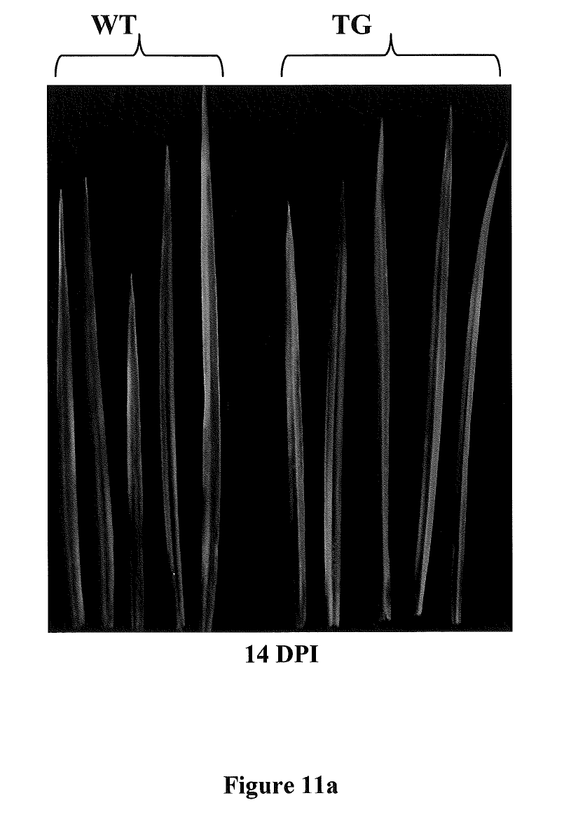

Construction of the Pen-1 gene and plant expression vectors. The two plant expression vectors in this work are presented in FIGS. 10a-b. Plasmid pHL016 (shown in FIG. 10a) contained only the single peptide sequence of Pen4-1, whereas plasmid pHL018 (shown in FIG. 10b) contained the single peptide sequence N terminally fused to an AP24 signal sequence (AP24) to obtain a chimeric Pen4 gene. For their expression in turfgrass, Pen4-1 genes were cloned between the maize ubiquitin (ubi) promoter and the nos terminator.

Plasmid pHL016 (pSBbarB/Ubi-Pen4-1). 147 by Pen4-1 coding sequence (with added start and stop codons) was amplified from pZEro-2:Pen4-1 (synthesized Pen4-1 from IDT, March 2007) by the following two primers: Pen4-ATG (with BamHI site added in the 5′ end) and Pen4-3 (with SphI and Sac1 sites added in the 5′ end). The amplified fragment was digested with BamHI and SacI site and ligated into the corresponding sites of pSBbarBUbiGUS (w/o BamHI, Hong Luo, 12/97) to replace the gusA coding sequence.

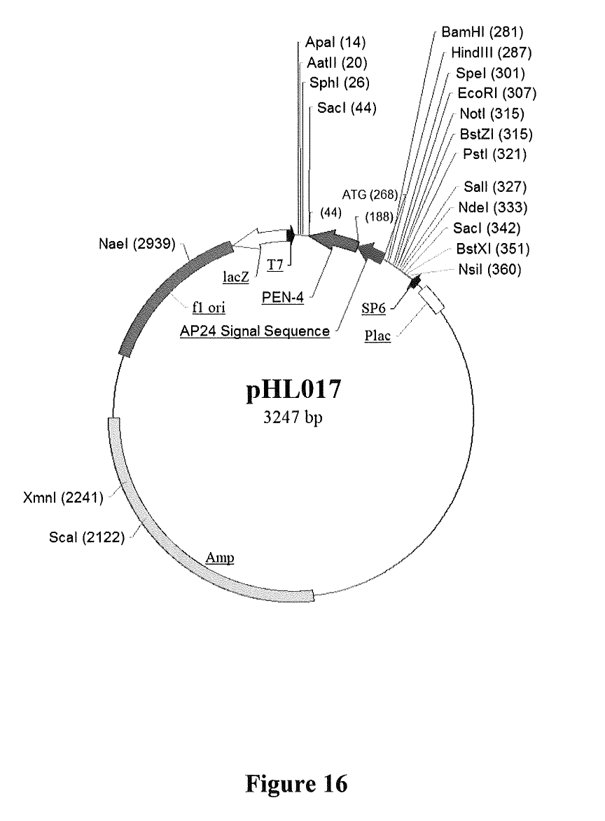

Plasmid pHL018 (pSBbarB/Ubi-AP24::Pen4-1). The amplified fragments of Pen4-1 with BamHI, SphI and Sad added were treated with Klenow (w/o dNTP), then digested with SphI and ligated into the Nco1 [flushed with Klenow(w/dNTP)]-Sph1 sites of the plasmid pGM-T-AP24 signal peptide (María et al, 2004) resulting in the plasmid pHL17 (FIG. 16). Correct sequence of amplified Pen4-1 and its frame fusion to the AP24 signal sequence were verified by sequencing. 241-bp AP24::Pen4-1 fusion gene released from pHL17 by BamHI and SacI digestions was ligated into the corresponding sites of pSBbarBUbiGUS to replace the gusA coding sequence.

Production of transgenic turfgrass plants. A commercial genotype of creeping bentgrass (Agrostis Stolonifera L.) Penn A-4 was used. Transgenic turfgrass lines expressing the Pen4-1 gene and the AP24::Pen4-1 fused gene were produced by Agrobacterium-mediated transformation of embryonic callus initiated from mature seeds essentially as previously described (Luo et al, 2004).

Plant DNA isolation and southern blot analysis. Genomic DNA was extracted from transgenic plants as previously described using a cetyltrimethylammonium bromide (CTAB) method (Luo et al, 2004). After digestion of the DNA with an appropriate amount of BamHI enzyme, DNAs were electrophoresed on 0.8% agarose gels, transferred onto nylon membranes, and hybridized to ³²P-labelled DNA probes of bar gene. Hybridization was carried out in Modified Church and Gilbert buffer at 65° C. (8). Membranes were washed in 0.1×SSC, 0.5% SDS at 65° C.

RNA isolation and northern blot analysis. Total RNA was isolated from the leaves of transgenic and control plants using Trizol (Invitrogen). RNAs were subjected to formaldehyde-containing agarose gel electrophoresis, and transferred onto Hybond-N nylon membranes. The DNA fragment coding for the Pen4-1 gene was used as probe. Hybridizations and membrane wash were performed as described by María et al (María et al, 2004).

In vitro plant leaf inoculation with R. solani and S. homoeocarpa. Transgenic plants were challenged with R. solani and S. homoeocarpa respectively, the most common cause of brown patch and dollar spot diseases in creeping bentgrass. Cultures were grown on potato dextrose agar at 25° C. for 3 days prior to inoculation. The inoculation was carried out in vitro in aseptic conditions. Ten top second expanded leaves from each plant were randomly chosen for inoculation. The leaves cut from plants were first washed with 70% ethanol and then washed with sterilized water. The leaves were put in petri dishes (150×15 mm) with 1% agar. An agar plug (d=3 mm) infested with mycelium of R. solani or S. homoeocarpa was placed on the bottom of the midrib of each leaf for inoculation. After inoculation, the petri dishes were put in a lighted growth chamber under a 14/10 h (day/night) photoperiod. Temperature and humidity conditions in the growth chamber were 28° C. and 70% RH. The brown patch disease development was calculated as the lesion length measured from the inoculated leaves after 2 days, 8 days and 14 days. The dollar spot disease development was calculated as the lesion length measured from the inoculated leaves after 2 days, 4 days and 7 days. The experiment was repeated three times.

In vivo direct plant inoculation with S. homoeocarpa and R. solani. The preparation of the fungi cultures S. homoeocarpa and R. solani and the inoculation on grass were conducted based on previously reported procedures (Wang et al, 2003; Dong et al, 2007). Selected transgenic lines based on molecular analysis were screened for resistance to S. homeocarpa. The wild type creeping bentgrass (Agrostis Stolonifera L. cv. Penn A-4) was included as control. Pots of each transgenic line or wild type cultivar were vegetatively replicated by transplanting approximately 20 mature shoots into plastic pots (9*10*10 cm size). Plantlets were grown in a greenhouse at 25-30° C. for 4 to 6 months. Fertilization, mowing and irrigation were carried out if needed. Fungal bioassays were conducted to assess levels of resistance among the transgenic lines towards S. homeocarpa and R. solani compared with control plants. The grasses were mowed prior to inoculation, and foliar coverage were estimated visually as the percentage of area per pot. The center of each pot was then inoculated with approximately 0.55 g of colonized inoculum by S. homeocarpa and 3 g of colonized inoculums by R. solani for one dose.

Plants inoculated with one dose of S. homoeocarpa were placed in plastic containers containing 4 cm of distilled water, and lightly misted with distilled water at 48-h intervals to maintain relative 100% humidity. The containers were placed inside a greenhouse set to maintain a diurnal cycle of 14 h light and 10 h dark. Three to four replicates of each transgenic line or wild type cultivar were used. Disease severity was visually estimated at 3, 5, 7 and 9 days post-inoculation using the Horsfall Barrett scale (Horsfall et al, 1945). Nine days later, the plants were taken out of the chamber and put in a growth room to recover for three weeks. Temperatures in the growth room were maintained at 22° C. in the light and 17° C. in the dark. The inoculation experiment was replicated three times.

Plants inoculated with a first dose of 3 g of rye grass seeds colonized by R. solani were placed in plastic containers containing 4 cm of distilled water, and lightly misted with distilled water at 48-h intervals to maintain humidity. The containers were placed inside a growth chamber to maintain a diurnal cycle of 14 h light and 10 h dark. The temperature and RH were 30° C. and 70% during day time, and 24° C. and 95% at night, respectively. After 14 days, disease was rated by measuring the total distance from the point of inoculation to the farthest point of the lesions extended. Then the plants were inoculated with a second dose of 3 g of colonized inoculums by R. solani to further observe the disease development in wild type and transgenic plants. After another 14 days, disease severity was visually estimated using the Horsfall Barrett scale (Horsfall et al, 1945). The inoculation experiment was replicated twice.

Statistical analysis. Both in vitro plant leaf inoculation and in vivo direct plant inoculation tests used randomized complete block design. Dollar spot and brown patch disease resistance was analyzed by ANOVA with the disease rating data and lesion length data. ANOVA was employed with Minitab 16 (Minitab Inc, PA, USA). CONTRAST statements were used to compare mean of each transgenic event to the mean of the wild type control plants.

Production of transgenic turfgrass containing the Pen4-1 gene. A total of 30 TO transgenic plants were obtained through Agrobacterium-mediated transformation from the disease resistance gene constructs. Among them, 25 contained the single Pen4-1 gene, and 5 had the AP24-Pen4-1 fused gene. The putative transgenic turfgrasses were first selected by herbicide resistance, for those plasmid constructs all contained bar genes.

Southern blot analysis of transgenic turfgrass. Southern analysis was performed on the 25 putative transgenic plants transformed with the single Pen4-1 gene and the transgenic nature of these plants was confirmed. The representative results are shown in FIG. 10c. The different sizes of the restricted transgene bands among the analyzed plants indicated stable integration of the transgenes at different loci in the creeping bentgrass genome, whereas the same sizes of the bands indicated that those transgenic plants might initiate from the same transformation event. Most of the transgenic plants were estimated to have only one copy of the transgene. Three of them were estimated to have 2-3 copies of the transgene.

Northern analysis of the transgenic plants. Northern blot analysis was carried out to detect the expression of the transgenes among the transgenic plants transformed with the single Pen4-1 gene proved by Southern blot. The results are shown in FIG. 10d. All the transgenic plants showed detectable transcript accumulation.

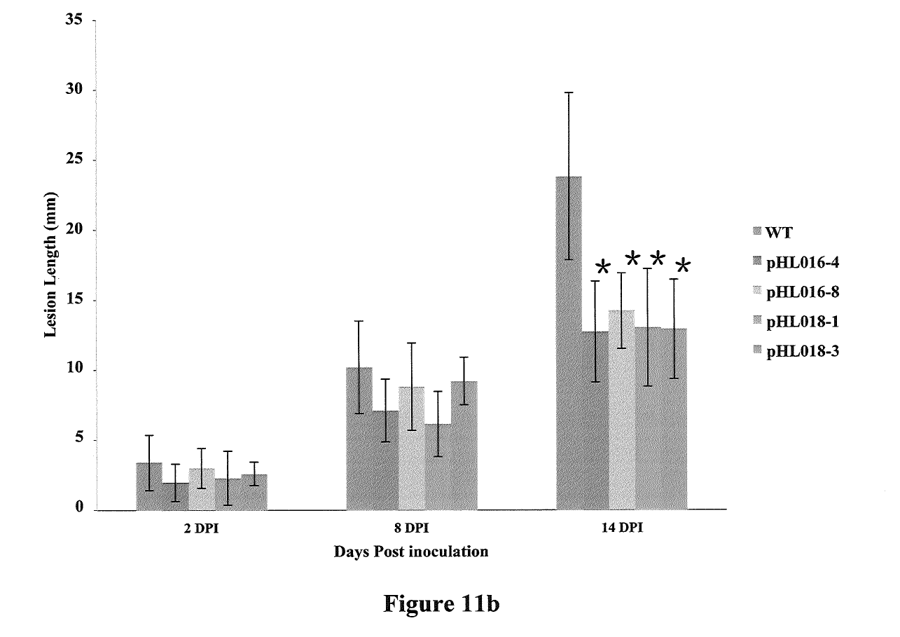

In vitro bioassays of brown patch disease resistance of transgenics. Wild type plants and transgenic plants were challenged with agar plug of R. solani in petri dishes. Transgenic lines pH1016-4, pHL016-8, pHL018-1 and pHL018-3 exhibited high resistance with the lesion length reduced by 42% to 48% compared with wild type control plants 14 days after inoculation (FIGS. 11a and 11b). Statistical analysis indicated that the lesion development among the different transgenic events was insignificant. However, the difference between wild type plants and the transgenic plants was significant on 14 DPI (FIG. 11b).

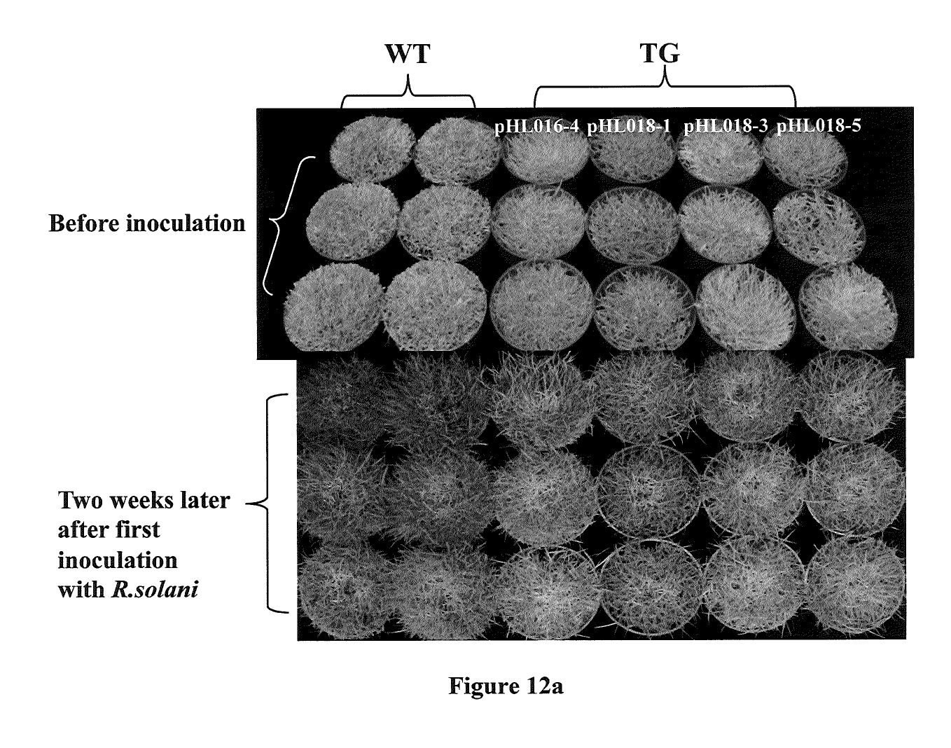

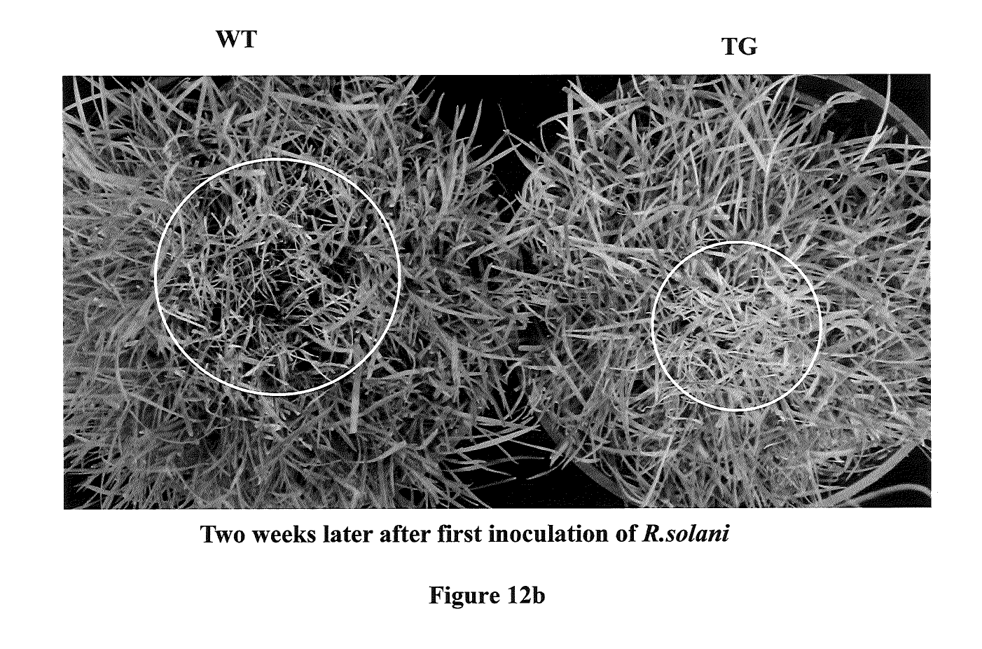

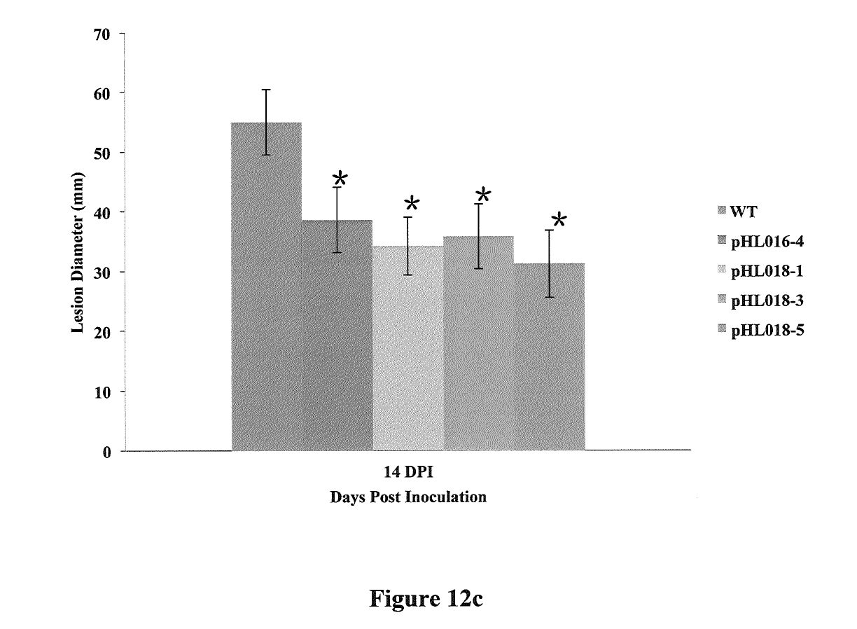

In vivo direct inoculation test for brown patch disease resistance. Transgenic line pHL016-4 (Pen4-1) and transgenic lines pHL018-1, pHL018-3, and pHL018-5 (AP24::Pen4-1) were challenged with a first dose (3 g) of rye seeds colonized by an R. solani isolate obtained from bentgrass in a replicated experiment under a controlled environment. 14 days after inoculation, transgenic lines all exhibited high resistance with the lesion diameter reduced from 30% to 43% (FIGS. 12a-c). Statistical analysis indicated that the disease development among the different transgenic lines was insignificant (FIG. 12c). However, there was a significant difference between wild type control plants and transgenic plants (FIG. 12c).

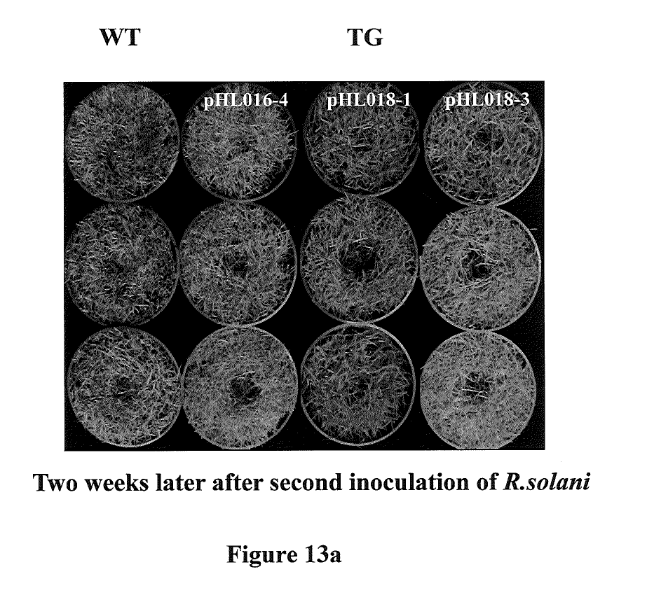

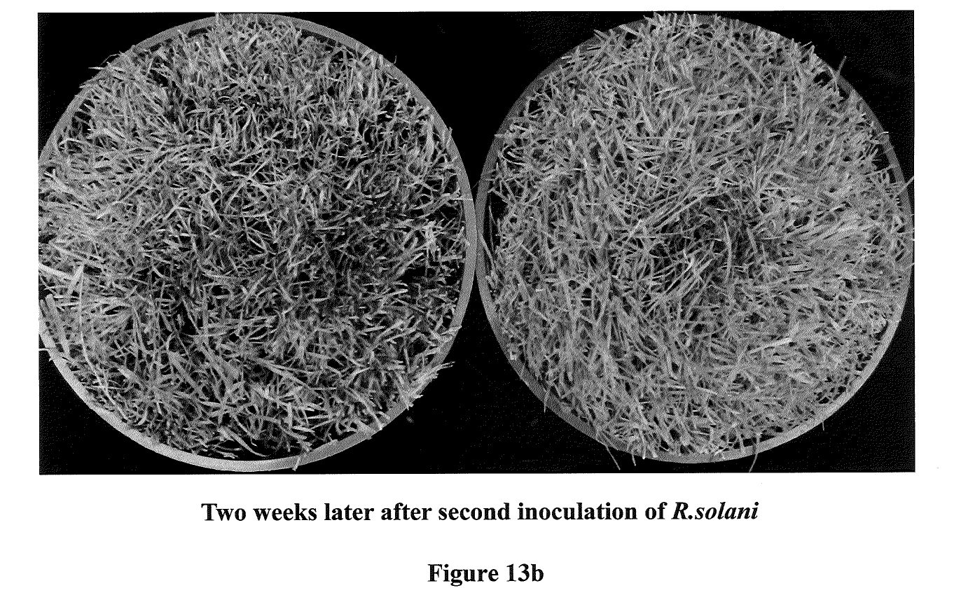

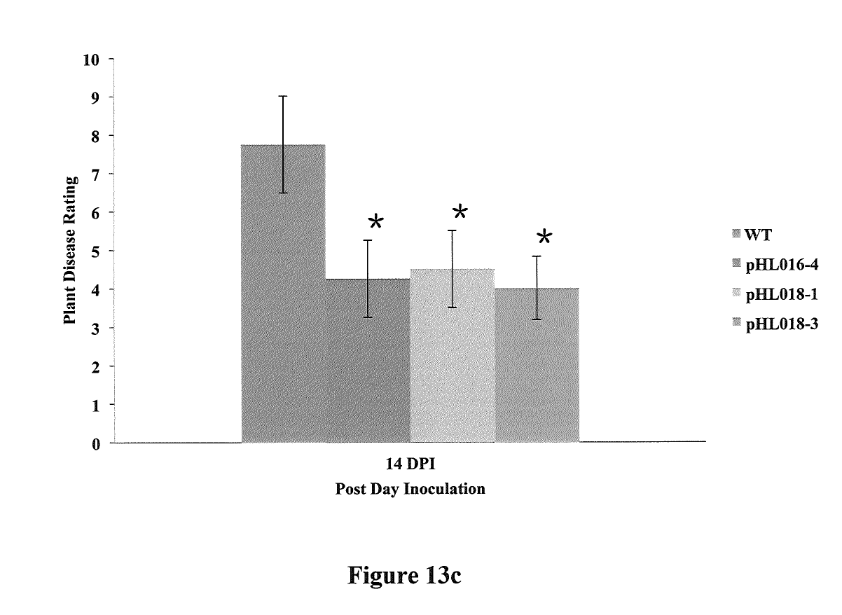

At this time point, a second dose (3 g) of rye grass seeds colonized by R. solani was inoculated on each pot of wild type plants and transgenic lines pHL16-4, pHL018-1 and pHL018-3. Two weeks later, large areas (around 75% to 95%) of turfgrass in the pots of wild type plants were infected. However, much smaller areas (around 25%) of turfgrass in the pots of transgenic plants were infected (FIGS. 13a-b). The disease ratings of transgenic plants were reduced from 41% to 44% compared with that of wild type plants (FIG. 13c). Statistical analysis indicated that the disease development among the different transgenic lines was insignificant (FIG. 13c).

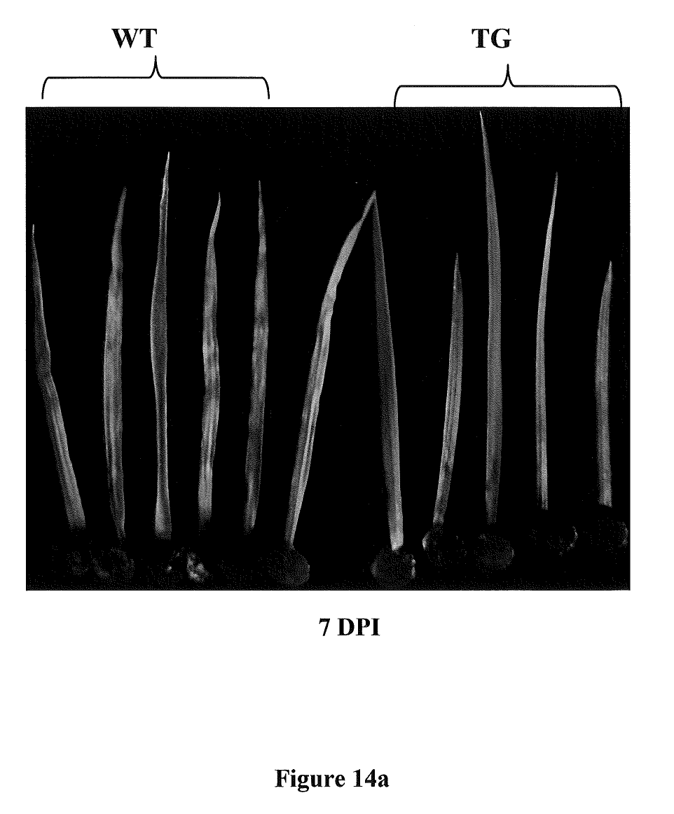

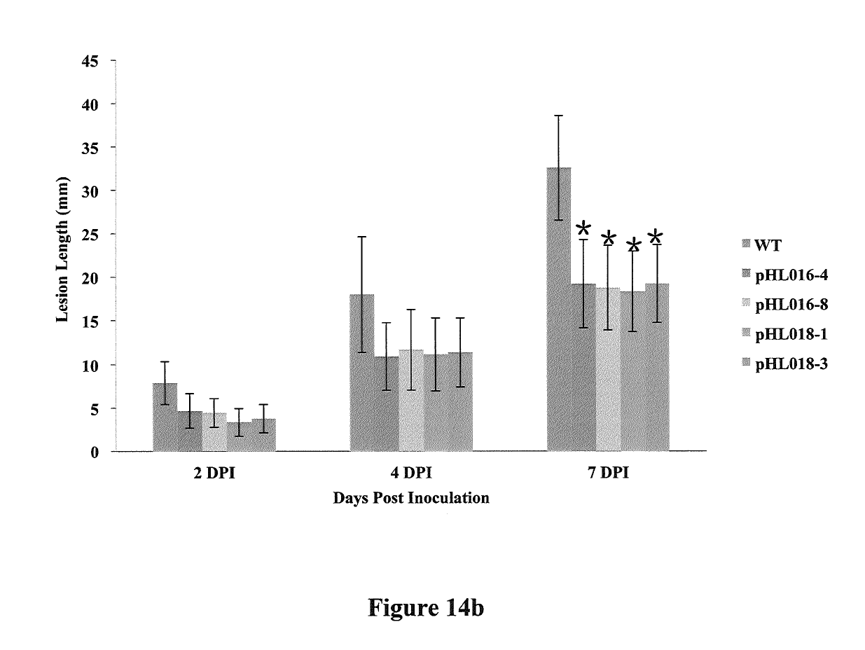

In vitro bioassays of dollar spot disease resistance of transgenics. Wild type plants and transgenic plants were challenged with an agar plug of S. homoeocarpa in petri dishes. Transgenic lines pH1016-4, pHL016-8, pHL018-1 and pHL018-3 exhibited high resistance with the lesion length reduced by 40% to 47% compared with wild type control plants 7 days after inoculation (FIGS. 14a-b). Statistical analysis indicated that the lesion development in the different transgenic events was insignificant. However, the difference between wild type plants and the transgenic plants was significant on 7 DPI (FIG. 14b).

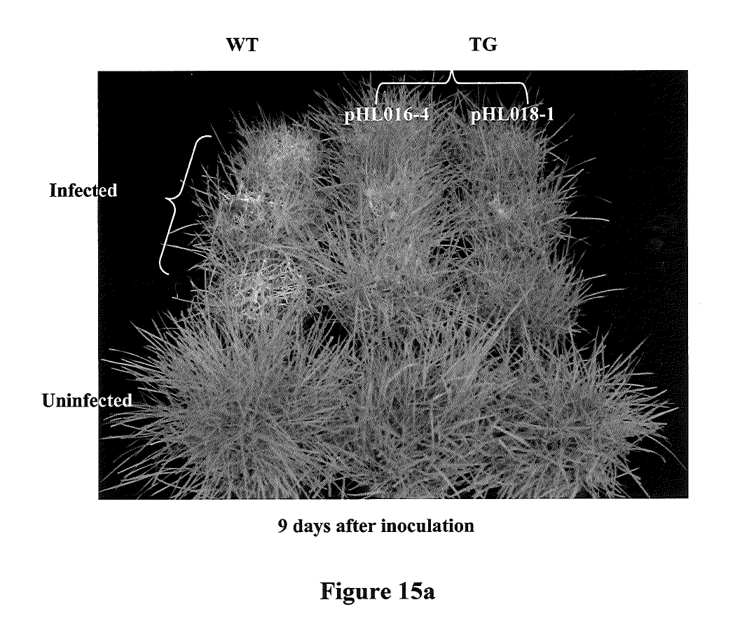

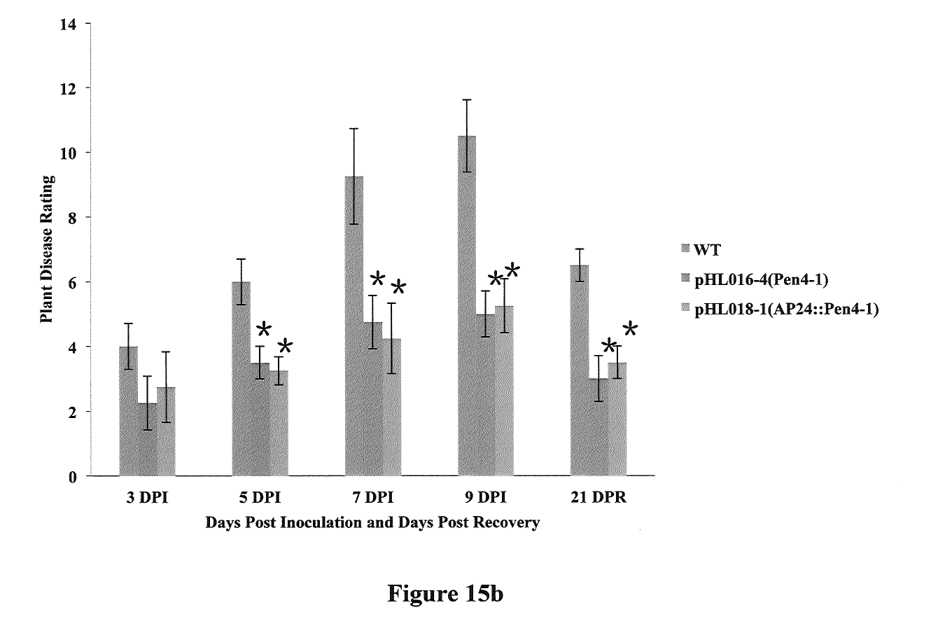

In vivo direct inoculation test for dollar spot disease resistance. Wild type plants and transgenic plants were challenged with a S. homoeocarpa isolate obtained from bentgrass in a replicated experiment under a controlled environment. Transgenic lines pHL016-4 (Pen4-1) and pHL018-1 (AP24::Pen4-1) exhibited high resistance with the disease rating reduced above 50% (FIGS. 15a-b). Statistical analysis indicated that the disease development in the transgenic plants was significantly delayed (FIG. 15b), and in the recovery phase the transgenic plants performed much better than wild type plants did. However, there was no significant difference between transgenic lines pHL016-4 (Pen4-1) and pHL018-1 (AP24::Pen4-1) (FIG. 15b).

Twenty five transgenic turfgrass lines constitutively expressing the Pen4-1 gene and five transgenic lines expressing the AP24::Pen4-1 fused gene were produced. All of them showed normal morphology and were fertile. The results here presented showed that the Pen4-1 gene was efficiently expressed.

### REFERENCES FOR EXAMPLE 1

1. Agrawal G K, Rakwal R, Jwa N S (2000) Rice (Oryza sativa L.) OsPR1b gene is phytohormonally regulated in close interaction with light signals. Biochem Biophys Res Commun 278:290-298.
2. Ainley M A (2000) The ecological impacts of transgenic crops on agroecosystem health. Ecosystem Health 6: 13-23.
3. Bagnaresi P, Moschella A, Beretta O, Vitulli F, Ranalli P, Perata P (2007) Heterologous microarray experiments allow the identification of the early events associated with potato tuber cold sweetening. BMC Genomics 9:176.
4. Baea T W, Vanjildorjb E, Songb S Y, Nishiguchia S, Yangc S S, Songa I J, Chandrasekhara T, Kangd T W, Kimc J I, Kohe Y J, Parkf S Y, Leeg J, Leeh Y E, Ryui K H, Riva K Z, Songa P S, Leea H Y (2008) Environmental Risk Assessment of Genetically Engineered Herbicide-Tolerant Zoysia japonica. J Environ Qual 37:207-218
5. Becher M, Talke I N, Krall L, Kramer U (2004) Cross-species microarray transcript profiling reveals high constitutive expression of metal homeostasis genes in shoots of the zinc hyperaccumulator Arabidopsis halleri. The Plant Journal 37:251-268.
6. Belanger F C, Bonos S, and Meyer W A (2004) Dollar spot resistant hybrids between creeping bentgrass and colonial bentgrass. Crop Sci 44:581-586.
7. Biezen V D E A (2001) Quest for antimicrobial genes to engineer disease-resistant crops. Trends Plant Sci 6:89-91.
8. Bomblies K, Wang R L, Ambrose B A, Schmidt R J, Meeley R B, Doebley J (2003) Duplicate FLORICAULA/LEAFY homologs zfl1 and zfl2 control inflorescence architecture and flower patterning in maize. Development 130(11):2385-95.
9. Cao H, Li X, Dong X (1998) Generation of broad-spectrum disease resistance by overexpression of an essential regulatory gene in systemic acquired resistance. Proc Natl Acad Sci USA 95: 6531-6536.
10. Chakraborty N, Chang T, Casler M D, and Jung G (2006) Response of Bentgrass Cultivars to Sclerotinia homoeocarpa Isolates Representing 10 Vegetative Compatibility Groups. Crop Sci 46(3): 1237-1244.
11. Chen, C. Y. Pan and C. M. Kuo (2004), Molecular cloning and sequencing of shrimp (Penaeus monodon) penaeidin-5 cDNA, Fish Shellfish Immunol 16:665-670.
12. Coen E S. Romero J M, Doyle S, Elliott R, Murphy G, Carpenter R (1990) floricaula: a homeotic gene required for flower development in Antirrhinum majus. Cell 63:1311-1322.
13. Cuthbertson B J, Millesbach E E, and Gross P S (2006). Discovery of Synthetic Penaeidin activity against Antibiotic-resistant fungi. Chem Biol Drug Des 68:120-127.
14. Cuthbertson B J, Yang Y, Bachere E, Bullesbach E E, Gross P S, and Aumelas A (2005) Solution Structure of Synthetic Penaeidin-4 with Structural and Functional Comparisons with Penaeidin-3 J Biol Chem 280(16): 16009-16018.
15. Daniell H (2002) Molecular strategies for gene containment in transgenic crops. Nat Biotechnol 20:581-586.
16. Destoumieux D, Bulet P, Strub J, Van Dorsselaer A and Bachere E (1999) Recombinant expression and range of activity of penaeidins, antimicrobial peptides from penaeid shrimp. Eur J Biochem 266:335-346.
17. Destoumieux D, Muñoz M, Bulet P and Bachere E (2000) Penaeidins, a family of antimicrobial peptides from penaeid shrimp (Crustacea, Decapoda). Cell Mol Life Sci 57: 1260-1271
18. Destoumieux D, Munoz M, Cosseau C, Rodriguez J, Bulet P, Comps M, Bachere E Penaeidins (2000) antimicrobial peptides with chitin-binding activity, are produced and stored in shrimp granulocytes and released after microbial challenge. J Cell Sci 113: 461-469.
19. Gocal G F W, King R W, Blundell C A, Schwartz O M, Andersen C H, Weigel D (2001) Evolution of Floral Meristem Identity Genes. Analysis of Lolium temulentum Genes Related to APETALA1 and LEAFY of Arabidopsis. Plant Physiol 125(4): 1788-1801.
20. Guo Z F, Bonos S, MeyerWA, Day P R & Belanger F C (2003) Transgenic creeping bentgrass with delayed dollar spot symptoms. Mol Breed 11: 95-101.
21. Gu Y Q, Wildermuth M C, Chakravarthy S, Loh Y T, Yang C, He X, Han Y, Martin G B (2002) Tomato transcription factors pti4, pti5, and pti6 activate defense responses when expressed in Arabidopsis. Plant Cell 14: 817-831.
22. Hancock R E W, Sahl H G (2006) Antimicrobial and host-defense peptides as new anti-infective therapeutic strategies. Nat Biotechnol 24:1551-57.
23. Hancock R E W (2005) Mechanisms of action of newer antibiotics for Gram-positive pathogens. Lancet Infect Dis 5:209-218.
24. Horsfall J G, Barratt R W (1945) An Improved Grading System for Measuring Plant Disease. Phytopathology 35:655.
25. Jwa N S, Agrawal G K, Rakwal R, Park C H, Agrawal V P (2001) Molecular cloning and characterization of a novel jasmonate inducible pathogenesis-related class 10 protein gene, JIOsPR10, from rice (Oryza sativa L.) seedling leaves. Biochem Biophys Res Commun 286:973-81
26. Keymanesh K, Soltani S and Sardari S (2009) Application of antimicrobial peptides in agriculture and food industry. World J Microbiol Biotechnol 25:933-944.
27. Kim Y J, Martin G B (2004) Molecular mechanisms involved in bacterial speck disease resistance of tomato. Plant Pathol J 20:7-12.
28. Koo J C, Chun H J, Park H C, Kim M C, Koo Y D, Koo S C, Ok H M, Park S J, Lee S H, Yun D J, Lim C O, Bahk J D, Lee S Y, Cho M J (2002) Over-expression of a seed specific hevein-like antimicrobial peptide from Pharbitis nil enhances resistance to a fungal pathogen in transgenic tobacco plants. Plant Mol Biol 50:441-452.
29. Kyozuka J, Konishi S, Nemoto K, Izawa T, Shimamoto K (1998) Down-regulation of RFL, the FLO/LFYhomolog of rice, accompanied with panicle branch initiation. PNAS 95:1979-1982.
30. Lee D G, Shin S Y, Kim D H, Seo M Y, Kang J H, Lee Y, Kim K L and Hahm K S (1999), Antifungal mechanism of a cysteine-rich antimicrobial peptide, Ib-AMP1, from Impatiens balsaminaagainst Candida albicans. Biotechnol Lett 21:1047-1050.
31. Lee S C, Hwang B K (2005.) Induction of some defense-related genes and oxidative burst is required for the establishment of systemic acquired resistance in Capsicum annum. Planta 221:790-800.
32. Lee S C, Hwang I S, Choi H W, Hwang B K (2009) Involvement of the Pepper Antimicrobial Protein CaAMP1 Gene in Broad Spectrum Disease Resistance. Plant Physiology 148:1004-102.
33. Li Q S, Lawrence C B, Xing H Y, Babbitt R A, Bass W T, Maiti I B (2001), Everett N P Enhanced disease resistance conferred by expression of an antimicrobial magainin analog in transgenic tobacco. Planta 212: 635-639.
34. Liu C A, Zhong H, Vargas J, Penner D and Sticklen M (1998), Prevention of fungal diseases in transgenic, bialaphos- and glufosinate-resistant creeping bentgrass (Agrostis palustris). Weed Sci 46 :139-146.
35. Liu G, Holub E B, Alonso J M, Ecker J R, Fobert P R (2005) An Arabidopsis NPR1-like gene, NPR4, is required for disease resistance. Plant J 41: 304-318.
36. Loon V L C, Rep M, Pieterse C M J. 2006. Significance of inducible defense-related proteins in infected plants. Annu Rev Phytopathol 44:135-62.
37. Luo H, Hu Q, Nelson K, Longo C, Kausch A P, Chandlee J M, Wipff J K, Fricker C R (2004) Agrobacterium tumefaciens-mediated creeping bentgrass (Agrostis stolonifera L.) transformation using phosphinothricin selection results in a high frequency of single-copy transgene integration. Plant Cell Rep 22: 645-652.
38. Luo H, Kausch A P, Hu Q, Nelson K, Wipff J K, Fricker C R, Owen T P, Moreno M A, Lee J Y, Hodges T K (2005) Controlling Transgene Escape in GM Creeping Bentgrass. Mol Breeding 16:185-188.
39. Ma Z, Michailides T J (2005) Advances in understanding molecular mechanisms of fungicide resistance and molecular detection of resistant genotypes in phytopathogenic fungi. Crop Prot 24:853-863.
40. Mishina T E, Zeier J (2007) Pathogen-associated molecular pattern recognition rather than development of tissue necrosis contributes to bacterial induction of systemic acquired resistance in Arabidopsis. Plant J 50:500-513.
41. Montesinos E (2007) Antimicrobial peptides and plant disease control. FEMS Microbial Lett 270(1):1-11.
42. Muñoz M, Vandenbulcke F, Saulnier D, Bachére E (2002) Expression and distribution of penaeidin antimicrobial peptides are regulated by haemocyte reactions in microbial challenged shrimp. Eur J Biochem 269: 2678-2689.
43. Osborn, R W, De Samblanx, G W, Thevissen K, Goderis I, Torrekens S, Van Leuven F, Attenborough S, Rees S B, Broekaert W F (1995) Isolation and characterisation of plant defensins from seeds of Asteraceae, Fabaceae, Hippocastanaceae and Saxifragaceae. FEBS Lett 368: 257-262.
44. Otvos Jr L (2000) Antibacterial peptides isolated from insects. J. Peptide Sci. 6, 497-511.
45. Otvos Jr L, Rogers M, Consolvo P, Condie B, Lovas S, Bulet P and Blaszczyk-Thurin M (2000) Interaction between heat shock proteins and antimicrobial peptides. Biochemistry 46: 14150-14159.
46. Park J M (2005) The hypersensitive response: a cell death during disease resistance. Plant Pathol J 21:99-101.
47. Patel S U, Osborn R, Rees S and Thornton J M (1998), Structural studies of Impatiens balsamina antimicrobial protein (Ib-AMP1). Biochemistry 37:983-990.
48. Ponti D, Mangoni M L, Mignogna G, Simmaco M, Barra D (2003) An amphibian antimicrobial peptide variant expressed in Nicotiana tabacum confers resistance to phytopathogens. Biochem J 370:121-127.
49. Poethig R S (1990) Phase Change and the Regulation of Shoot Morphogenesis in Plants. Science 250:923-930.
50. Poethig R S (2003) Phase Change and the Regulation of Developmental Timing in Plants. Science 301: 334-336.
51. Qu R D, Luo H, Meier V D (2008) Turfgrass, In: Compendium of Transgenic Crop Plants: Transgenic Plantation Crops, Ornamentals and Turf Grasses, Kole C, Hall T C (eds), Blackwell Publishing, Oxford, UK, pp. 177-218.
52. Rekha, Naik S N, Prasad R (2006) Pesticide residue in organic and conventional food-risk analysis. J Chem Health Saf 13:12-19.
53. Reichman J R, Watrud L S, Lee E H, Burdick C A, Bollman M A, Storm M J, King G A, Mallory-Smith C (2006) Establishment of transgenic herbicide-resistant creeping bentgrass (Agrostis stolonifera L.) in nonagronomic habitats. Mol Ecol 15:4243-4255.
54. Sang Y, Blecha F (2009) Antimicrobial peptides and bacteriocins: alternatives to traditional antibiotics Animal Health Research Reviews 9(02):227.
55. Schaefer S C, Gasic K, Cammue B, Broekaert W, van Damme E J, Peumans W J, Korban S S (2005) Enhanced resistance to early blight in transgenic tomato lines expressing heterologous plant defense genes. Planta 222:858-866.
56. Selitrennikoff C P (2001) Antifungal proteins. Appl Environ Microbiol 67:2883-2894 Snow A A (2002) Transgenic crops-why gene flow matters. Nat Biotechnol 20(6):542.
57. Tailor R H, Acland D P, Attenborough S, Cammue B P, Evans I J and Osborn R W (1997), A novel family of small cysteine-rich antimicrobial peptides from seed of Impatiens balsamina is derived from a single precursor protein, J Biol Chem 272:24480-24487.
58. The Gene Ontology Consortium (2001) Creating the Gene Ontology Resource: Design and Implementation. Genome Res 11:1425-1433.
59. Vizioli J, Salzet M (2002b) Antimicrobial peptides from animals: focus on invertebrates. Trends Pharmacol Sci 23:494-496.
60. Wang Y, Browning M, Ruemmele B A, Chandlee J M, Kausch A P and Jackson N (2003) Glufosinate reduces fungal diseases in transgenic glufosinate-resistant bentgrasses (Agrostis spp.). Weed Sci 51(1):130-137.
61. Weigel D, Alvarez J, Smyth D R, Yanofsky M F, Meyerowitz E M (1992) Leafy controls floral meristem identity in Arabidopsis. Cell 69:843-859.
62. Yang Y, Poncet J, Garnier J, Zatylny C, Bachére E and Aumelas A (2003) Solution structure of the recombinant penaeidin-3, a shrimp antimicrobial peptide. J Biol Chem 278:36859-36867.
63. Yount N Y, Yeaman M R (2005) Immunocontinuum: perspectives in antimicrobial peptide mechanisms of action and resistance. Protein Pept Lett 12:49-67.
64. Zakharchenko N S, Rukavtsova E B, Gudkov A T, Buryanov Ya I (2005) Enhanced resistance to phytopathogenic bacteria in transgenic tobacco plants with synthetic gene of antimicrobial peptide cecropin P1. Russ J Genet 41:1445-1452.
65. Zapiola M, Campbell C, Butler M, Mallory-Smith C (2008) Escape and Establishment of Transgenic Glyphosate-resistant Creeping Bentgrass (Agrostis stolonifera) in Oregon, USA: A 4-year Study. J. Applied Ecology 45:486-494.
66. Zasloff M (2002) Antimicrobial peptides of multicellular organisms. Nature 415:389-395.
67. Sharma A, Sharma R, Imamura M, Yamakawa M, Machii H (2000) Transgenic expression of cecropin B, an antibacterial peptide from Bombyx mori, confers enhanced resistance to bacterial leaf blight in rice. FEBS Lett 484:7-11.
68. Chiou P P, Lin C M, Perez L, Chen T T (2002) Effect of cecropin B and a synthetic analogue on propagation of fish viruses in vitro. Mar Biotechnol 4:294-302.
69. Coca M, Penas G, Gomez J, Campo S, Bortolotti C, Messeguer J, Segundo B S (2006) Enhanced resistance to the rice blast fungus Magnaporthe grisea conferred by expression of a cecropin A gene in transgenic rice. Planta 223:392-406.
70. Zhang J X, Zhang S F, Wang T D, Guo X J, Hu R L (2007) Mammary gland expression of antibacterial peptide genes to inhibit bacterial pathogens causing mastitis. J Dairy Sci 90:5218-5225
71. Ponti D, Mangoni M L, Mignogna G, Simmaco M, Barra D (2003) An amphibian antimicrobial peptide variant expressed in Nicotiana tabacum confers resistance to phytopathogens. Biochem J 370:121-127.
72. Lu G (2003) Engineering Sclerotinia sclerotiorum resistance in oilseed crops. Afr J Biotechnol 2:509-516
73. Gao A G, Hakimi S M, Mittanck C A, Wu Y, Woerner B M, Stark D M, Shah D M, Liang J, Rommens C M (2000) Fungal pathogen protection in potato by expression of a plant defensin peptide Nat Biotechnol. 18(12):1307-10.
74. Koo J C, Chun H J, Park H C, Kim M C, Koo Y D, Koo S C, Ok H M, Park S J, Lee S H, Yun D J, Lim C O, Bahk J D, Lee S Y, Cho M J (2002) Over-expression of a seed specific hevein-like antimicrobial peptide from Pharbitis nil enhances resistance to a fungal pathogen in transgenic tobacco plants. Plant Mol Biol 50:441-452.
75. Schaefer S C, Gasic K, Cammue B, Broekaert W, van Damme E J, Peumans W J, Korban S S (2005) Enhanced resistance to early blight in transgenic tomato lines expressing heterologous plant defense genes. Planta 222:858-866.
76. Kazan K, Rusu A, Marcus J P, Goulter K C, Manners J M (2002) Enhanced quantitative resistance to Leptosphaeria maculans conferred by expression of a novel antimicrobial peptide in canola (Brassica napus L.). Mol Breed 10:63-70
77. Wang Y, Nowak G, Culley D, Hadwiger L A, Fristensky B (1999) Constitutive expression of pea defense gene DRR206 confers resistance to blackleg (Leptosphaeria maculans) disease in transgenic canola (Brassica napus). Mol Plant Microbe Interact 12:410-418.
78. Reuveni M, Cohen H, Zahavi T, Venezian A (2000) Polar-a potent Polyoxin B compound for controlling powdery mildews in apple and nectarine trees, and grapevines. Crop Prot 19:393-399.
79. Arakawa T (2003) Chitin synthesis-inhibiting antifungal agents promote nucleopolyhedrovirus infection in silkworm, Bombyx mori (Lepidoptera: Bombycidae) larvae. J Invertebr Pathol 83:261-263.
80. Delves-Broughton J (2005) Nisin as a food preservative. Food Aust 57:525-527.
81. María Coca, Cristina Bortolotti, Mar Rufat, Gisela Peñas, Ramón Eritja, Didier Tharreau, Alvaro Martinez del Pozo Joaquima, Messeguer and Blanca San Segundo (2004) Transgenic Rice Plants Expressing the Antifungal AFP Protein from Aspergillus Giganteus Show Enhanced Resistance to the Rice Blast Fungus Magnaporthe Grise. Plant Molr Biol 54: 245-259.
82. Herbers K., Wilke I. and Sonnewald U. 1995. A thermostable xylanase from Clostridium thermocellum expressed al high levels in the apoplast of transgenic tobacco has no detrimental effects and is easily purified. Bio/technology 13: 63-66.
83. Sonia Wirth, Gabriela Calamante, Alejandro Mentaberry, Leonardo Bussmann, Mariano Lattanzi, Lino Barafiao and Fernando Bravo-Almonacid (2004) Expression of active human epidermal growth factor (hEGF) in tobacco plants by integrative and non-integrative systems Mol. Breeding 13(4): 1380-3743.
84. Leite A. Kemper E., da Silva M. J., Luchessi A. D., Siloto R. P., Bonaccorsi E. D., El-Dorry H. F. and Arruda P. 2000. Expression 34 of correctly processed human growth hormone in seeds of transgenic tobacco plants. Mol. Breed. 6: 47-53.
85. Ottoboni L M M, Leite A, Yunes J A, Targon M L P N, de Souza Filho G A, Arruda P: Sequence analysis of 22 kDa-like_coixin and their comparison with homologous zein and kafirin genes reveals highly conserved protein structure and regulatory elements. Plant Mol Biol 21: 765-778 (1993).
86. Francois I E J A, De Bolle M F C, Dwyer G, Goderis I J W M, Woutors P F J, Verhaert P D, Proost P, Schaaper W M M, Cammue B P A and Broekaert W F (2002) Transgenic expression in Arabidopsis of a polyprotein construct leading to production of two different antimicrobial proteins. Plant Physiol 128: 1346-1358.
87. Sabelle E. J. A. Francois, Geoff I. Dwyera, Miguel F. C. De Bollea, Inge J. W. M. Goderisa, W. Van Hemelrijck, Paul Proost, Piet Woutersa, Willem F. Broekaert, and Bruno P. A. Cammue (2002) Processing in transgenic Arabidopsis thaliana plants of polyproteins with linker peptide variants derived from the Impatiens balsamina antimicrobial polyprotein precursor. Plant Physiology and Biochemistry 40: 871-879.
88. Ma C and Mitra A (2002) Expressing multiple genes in a single open reading frame with the 2A region of foot-and-mouth disease virus as a linker. Mol Breeding 9: 191-199.
89. Brian Miki and Sylvia McHugh (2003) Selectable marker genes in transgenic plants: applications, alternatives and biosafety 107:(193-232)
90. María Coca, Cristina Bortolotti, Mar Rufat, Gisela Peñas, Ramón Eritja, Didier Tharreau, Alvaro Martinez del Pozo Joaquima, Messeguer and Blanca San Segundo (2004) Transgenic rice plants expressing the antifungal AFP protein from Aspergillus giganteus show enhanced resistance to the rice blast fungus Magnaporthe grise. Plant Molecular Biology 54: 245-259.
91. Mazodier, P. Cossart, E. Giraud and F. Gasser (1985), Completion of the nucleotide sequence of the central region of Tn5 confirms the presence of three resistance genes Nucleic Acids Res. 13:195-205.
92. Worobo et al. (1995) A signal peptide secretion-dependent bacteriocin from Carnobacterium divergens. J. Bacteriol. 177(11): 3141-3149.

### REFERENCES FOR EXAMPLE 2

Bachere, E., Destoumieux, D. and Bulet, P. (2000) Aquaculture 191, 71-88
Chai, B., Maqbolol, S. B., Hajela, R. K., Green, D., Vargas, Jr. J. M., Warkentin, D., Sabzikar, R. and Sticklen, M. B. (2002) Plant Sci 163, 183-193.
Chen, C. Y. Pan and C. M. Kuo (2004), “Molecular cloning and sequencing of shrimp (Penaeus monodon) penaeidin-5 cDNA,” Fish Shellfish Immunol 16:665-670.
Church, G. M. and Gilbert, W. (1984) Proc Natl Acad Sci USA 81, 1991-1995
Cuthbertson, B. J., Büllesbach, E. E. and Gross, P. S. (2006) Chem Biol Drug Des 68, 120-127.
Cuthbertson B J, Yang Y, Bachere E, Büllesbach E E, Gross P S, and Aumelas A (2005) “Solution Structure of Synthetic Penaeidin-4 with Structural and Functional Comparisons with Penaeidin-3” J Biol Chem 280(16): 16009-16018.
Destoumieux D, Bulet P, Strub J, Van Dorsselaer A and Bachere E (1999) “Recombinant expression and range of activity of penaeidins, antimicrobial peptides from penaeid shrimp” Eur J Biochem 266:335-346.
Destoumieux D, Muñoz M, Bulet P and Bachere E (2000) “Penaeidins, a family of antimicrobial peptides from penaeid shrimp (Crustacea, Decapoda).” Cell Mo Life Sci 57: 1260-1271.
Dong S J, Tredway L P, Shew H D, Wang G L, Sivamani E, Qu R D (2007) “Resistance of transgenic tall fescue to two major fungal diseases” Plant Sci 173 (5): 501-509
Horsfall J G, Barratt R Wl (1945) “An Improved Grading System for Measuring Plant Disease” Phytopathology 35:655.
Li Q S, Lawrence C B, Xing H Y, Babbitt R A, Bass W T, Maiti I B and Everett N P (2001) 212, 635-639
Luo H, Hu Q, Nelson K, Longo C, Kausch A P, Chandlee J M, Wipff J K and Fricker C R (2004) Plant Cell Rep 28, 1793-1804
Luo, H., Van Coppenolle, B., Seguin, M. and Boutry, M. (1995) Mol Breeding 1, 51-63
María C, Cristina B, Mar R, Gisela P, Ramón E, Didier T, Alvaro M, Joaquima M and Blanca S S (2004) Plant Mol Biol 54, 245-259
Muñoz M, Vandenbuicke F, Saulnier D, Bachère E (2002) “Expression and distribution of penaeidin antimicrobial peptides are regulated by haemocyte reactions in microbial challenged shrimp” Eur J Biochem 269: 2678-2689.
Wang Y, Browning M, Ruemmele B A, Chandlee J M, Kausch A P and Jackson N (2003) Glufosinate reduces fungal diseases in transgenic glufosinate-resistant bentgrasses (Agrostis spp.). Weed Sci 51(1):130-137.
Yang Y, Poncet J, Garnier J, Zatylny C, Bachère E and Aumelas A (2003) “Solution structure of the recombinant penaeidin-3, a shrimp antimicrobial peptide” J Biol Chem 278:36859-36867.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

That which is claimed is:

1. A nucleic acid construct comprising:
a) a nucleotide sequence encoding PEN4-1;
b) a nucleotide sequence encoding Ib-AMP4; and
c) a promoter operably associated with the nucleotide sequence of (a).

2. A nucleic acid construct comprising:
a) a nucleotide sequence encoding PEN4-1;
b) a nucleotide sequence encoding Ib-AMP4; and
c) a promoter operably associated with the nucleotide sequence of (b).

3. The nucleic acid construct of claim 1, further comprising a termination sequence.

4. The nucleic acid construct of claim 1, further comprising a signal peptide sequence.

5. The nucleic acid construct of claim 1, further comprising a linker peptide.

6. The nucleic acid construct of claim 1, further comprising a selectable marker sequence.

7. The nucleic acid construct of claim 1, comprising in the following order from 5′ to 3′:
a) a corn ubiquitin promoter;
b) an AP24 signal peptide sequence;
c) a nucleotide sequence encoding PEN4-1;
d) an IbAMP propeptide;
e) a nucleotide sequence encoding IbAMP-4;
f) a first nos sequence;
g) a rice ubiquitin promoter sequence;
h) a bar coding sequence; and
i) a second nos sequence.

8. The nucleic acid construct of claim 2, comprising in the following order from 5′ to 3′:
a) a corn ubiquitin promoter;
b) an AP24 signal peptide sequence;
c) a nucleotide sequence encoding IbAMP4;
d) an IbAMP propeptide;
e) a nucleotide sequence encoding PEN4-1;
f) a first nos sequence;
g) a rice ubiquitin promoter sequence;
h) a bar coding sequence; and
i) a second nos sequence.

9. The nucleic acid construct of claim 1, comprising in the following order from 5′ to 3′:
a) a CaMV 35S promoter sequence;
b) a FLO/LFY antisense sequence;
c) a GUS linker sequence;
d) a FLO/LFY sense sequence;
e) a first nos sequence;
f) a corn ubiquitin promoter;
g) an AP24 signal peptide sequence;
h) a nucleotide sequence encoding PEN4-1;
i) a second nos sequence;
j) a rice ubiquitin promoter sequence;
k) a bar coding sequence; and
l) a third nos sequence.

10. The nucleic acid construct of claim 2, comprising in the following order from 5′ to 3′:
a) aCaMV 35S promoter sequence;
b) a FLO/LFY antisense sequence;
c) a GUS linker sequence;
d) a FLO/LFY sense sequence;
e) a first nos sequence;
f) a corn ubiquitin promoter;
g) an AP24 signal peptide sequence;
h) a nucleotide sequence encoding Ib-AMP-4;
i) a second nos sequence;
j) a rice ubiquitin promoter sequence;
k) a bar coding sequence; and
l) a third nos sequence.

11. A transformed plant cell comprising the nucleic acid construct of claim 1.

12. A transformed plant cell comprising the nucleic acid construct of claim 2.

13. A transgenic plant comprising the nucleic acid construct of claim 1.

14. A transgenic seed from the transgenic plant of claim 13.

15. A method of producing a transgenic plant having increased resistance to bacterial and/or fungal infection, comprising:
a) transforming a cell of a plant with the nucleic acid construct of claim 1; and
b) regenerating the transgenic plant from the transformed plant cell, wherein the plant has increased resistance to bacterial and or fungal infection as compared with a plant that is not transformed with said nucleic acid construct.

16. A method of producing a transgenic plant having increased resistance to bacterial and/or fungal infection, comprising:
a) transforming a cell of a plant with the nucleic acid construct of claim 2; and
b) regenerating the transgenic plant from the transformed plant cell, wherein the plant has increased resistance to bacterial and or fungal infection as compared with a plant that is not transformed with said nucleic acid construct.

17. A method of producing a transgenic plant having increased resistance to bacterial and/or fungal infection, comprising:
a) transforming a cell of a plant with a nucleic acid construct comprising a nucleotide sequence encoding PEN4-1; and
b) regenerating the transgenic plant from the transformed plant cell, wherein the plant has increased resistance to bacterial and or fungal infection as compared with a plant that is not transformed with said nucleic acid construct.

18. A transgenic plant produced by the method of claim 15.

19. A transgenic plant produced by the method of claim 16.

20. A transgenic plant produced by the method of claim 17.
